(19) United States
(12) Patent Application Publication
(10) Pub. No.: US 2026/0122457 A1
(43) Pub. Date: Apr. 30, 2026

(54) SYSTEM AND METHOD OF INSTRUMENT COOPERATION WITH CELLULAR CONNECTIVITY

(71) Applicant: Industrial Scientific Corporation, Pittsburgh, PA (US)

(72) Inventors: Bryan James Pavlisko, Aliquippa, PA (US); Jason Christopher Kettler, Follansbee, WV (US); Joseph Shih, Houston, TX (US); Adrian Sirbu, Coraopolis, PA (US)

(21) Appl. No.: 19/402,055

(22) Filed: Nov. 26, 2025

Related U.S. Application Data
(63) Continuation of application No. PCT/US2025/040297, filed on Aug. 1, 2025.
(60) Provisional application No. 63/711,936, filed on Oct. 25, 2024.

(51) Int. Cl. H04W 4/38; G01N 33/00; H04L 67/12

(52) U.S. Cl. CPC H04W 4/38; G01N 33/0075; H04L 67/12

(57) ABSTRACT

A system includes a plurality of gas monitors, each gas monitor including a means for variable ambient air flow accommodation, a means for stacking groups of the plurality of gas monitors, and a means for operating the plurality of gas monitors as a cooperative monitoring group and for providing at least selected gas monitoring data to a remote device.

### CROSS-REFERENCE TO RELATED APPLICATIONS

This application claims priority to and is a continuation of International Patent Application Serial No. PCT/US2025/040297, filed on Aug. 1, 2025 (Attorney Docket No. ISCI-0052-WO).

International Patent Application Serial No. PCT/US2025/040297 claims the benefit of and priority to U.S. application Ser. No. 63/711,936, filed on Oct. 25, 2024 (Attorney Docket No. ISCI-0052-P01).

Each of the foregoing applications is incorporated herein by reference in its entirety for all purposes.

### BACKGROUND

Previously known area monitors are difficult to deploy in remote areas at scale and by hand, and were limited in the orientation of their placement thus restricting gas flow, compromising the fidelity with which the surrounding air is monitored, and increasing deployment times where the orientation of individual gas monitors needs to be implemented and checked during the deployment. Previously known area monitors required battery replacement during their deployment making long-term monitoring a difficulty, and further require a shutdown of the monitoring device during battery replacement operations.

### SUMMARY

The disclosure herein provides numerous benefits with respect to addressing the challenges of area deployment of monitoring units. Other benefits may also be present, and embodiments disclosed herein may not have certain or all of the described benefits. This disclosure supports at least the following benefits: rapid rollout and collection of mass deployments of area monitoring units with an increase in tolerance of final position during deployment, area monitoring units that can run for weeks without support, easy charging and battery replacement of area monitoring units including under challenging field conditions, and embodiments of area monitoring units that are self-sufficient (power and communications) without any supporting infrastructure. A person of skill in the art having the benefit of this disclosure will understand that the disclosed area monitoring units are especially suited for deployments that are rapid, temporary, large, and/or remote.

### BRIEF DESCRIPTION OF THE FIGURES

FIG. 1 is a depiction of two stacked monitoring units in accordance with example embodiments of the present disclosure.

FIG. 2A and FIG. 2B are depictions of a centralized charger and battery packs in accordance with example embodiments of the present disclosure.

FIG. 3 is a depiction of transportation and deployment of monitoring units in accordance with example embodiments of the present disclosure.

FIG. 4 is a depiction of deployment of monitoring units in accordance with example embodiments of the present disclosure.

FIG. 5 is a depiction of active monitoring of monitoring units in accordance with example embodiments of the present disclosure.

FIG. 6 is a depiction of field maintenance of a monitoring unit in accordance with example embodiments of the present disclosure.

FIG. 7 is a depiction of a perspective view of a monitoring unit in accordance with example embodiments of the present disclosure.

FIG. 8 is a depiction of a left side of a monitoring unit in accordance with example embodiments of the present disclosure.

FIG. 9 is a depiction of a right side of a monitoring unit in accordance with example embodiments of the present disclosure.

FIG. 10 is a depiction of a front side of a monitoring unit in accordance with example embodiments of the present disclosure.

FIG. 11 is a depiction of a back side of a monitoring unit in accordance with example embodiments of the present disclosure.

FIG. 12 is a depiction of a top side of a monitoring unit in accordance with example embodiments of the present disclosure.

FIG. 13 is a depiction of a bottom side of a monitoring unit in accordance with example embodiments of the present disclosure.

FIG. 14 is a depiction of a cavity of a monitoring unit in accordance with example embodiments of the present disclosure.

FIG. 15 is a depiction of a gas path of a monitoring unit in accordance with example embodiments of the present disclosure.

FIG. 16, FIG. 17, FIG. 18, FIG. 19, and FIG. 20 are depictions of a monitoring unit in accordance with example embodiments of the present disclosure.

FIG. 21 is a schematic block diagram of a monitoring unit in accordance with example embodiments of the present disclosure.

FIG. 22A is a schematic of a bottom portion of a gas monitor.

FIG. 22B is a schematic of a bottom portion of a gas monitor.

FIG. 22C is a schematic of a bottom portion of a gas monitor.

FIG. 23 is a schematic block diagram of a monitoring unit in accordance with example embodiments of the present disclosure.

FIG. 24A is a flow diagram of an example procedure of the present disclosure.

FIG. 24B is a flow diagram of an example procedure of the present disclosure.

FIGS. 25A, 25B, 25C, and 25D are schematics of a gas monitor with a curved engagement notch and slot.

### DETAILED DESCRIPTION

The present disclosure relates to a systems, methods, and apparatuses developed for serving monitoring needs across applications and industries, such as by providing form factors suited to the environments in which they are deployed and the needs of each application, all while maintaining existing safety capabilities, optimizing fleet size, reducing downtime, and simplifying transportation & deployment.

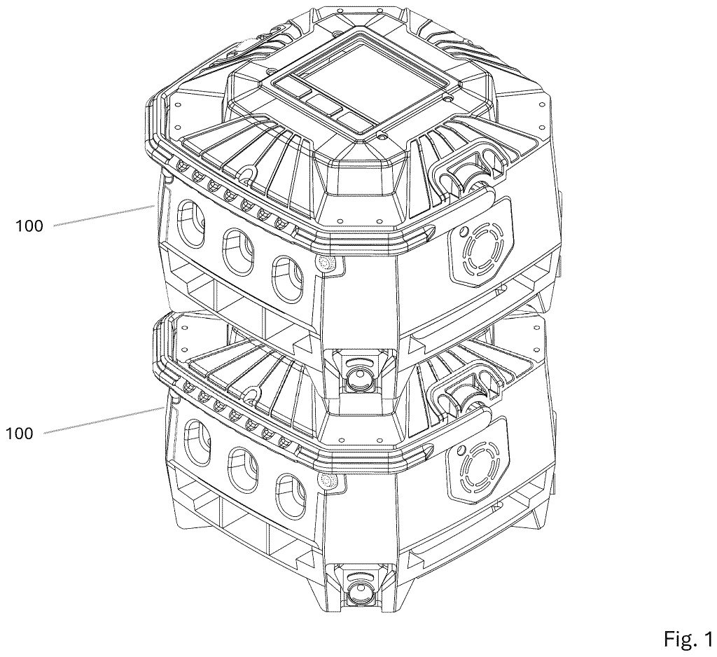

FIG. 1 illustrates two example stacked area monitoring units 100 in accordance with example embodiments. As shown in FIG. 1, and as described herein, area monitoring units 100 according to example embodiments may be stacked and locked together. Area monitoring units 100 in accordance with example embodiments may, for example, be used to monitor areas, such as work spaces, to detect hazardous conditions or other conditions, such as workplace safety conditions, which may require an alert. Areas may include, for example, those pertaining to oil and gas, chemical, shipbuilding, fire services, pulp and paper, and water and wastewater.

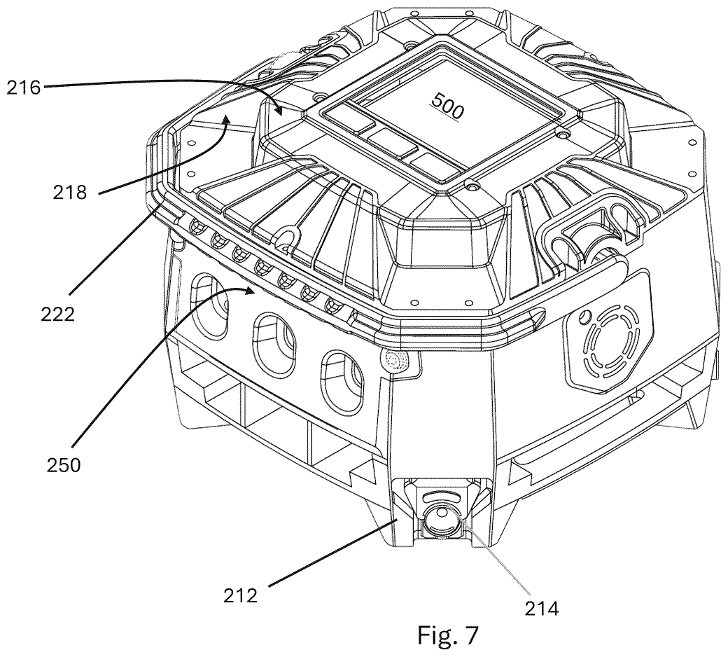

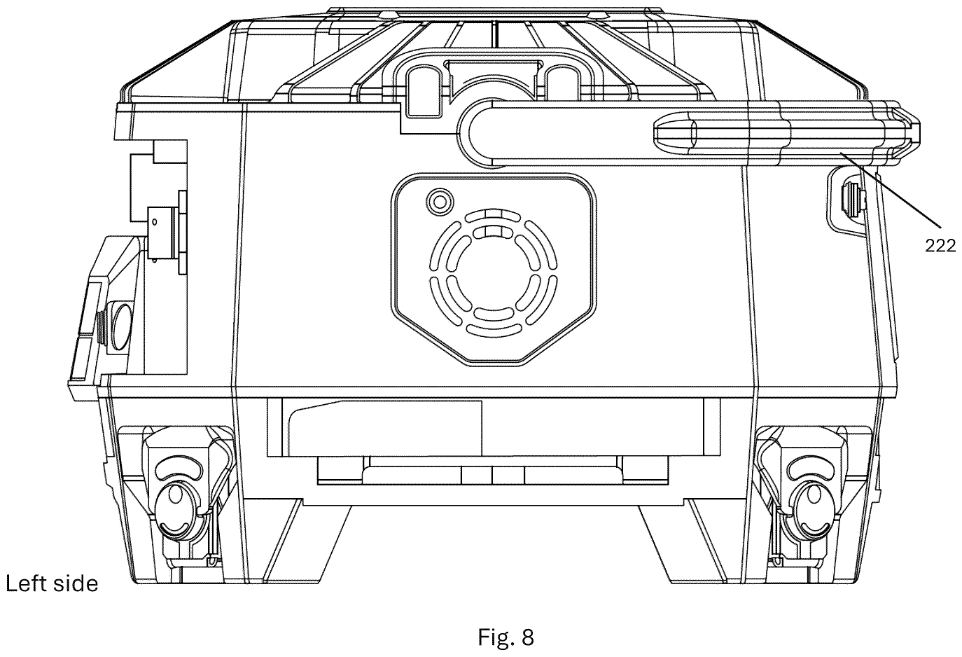

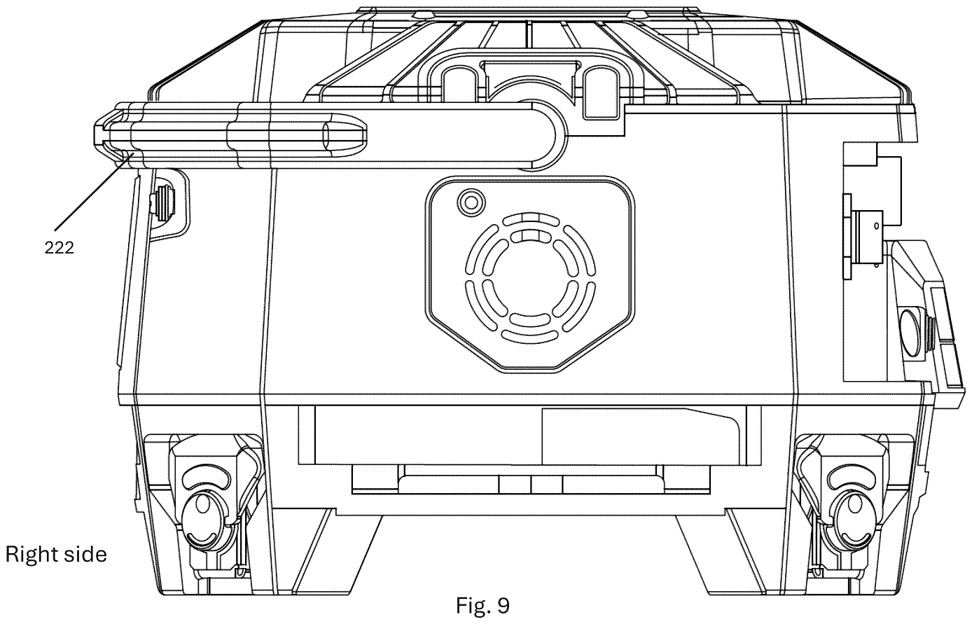

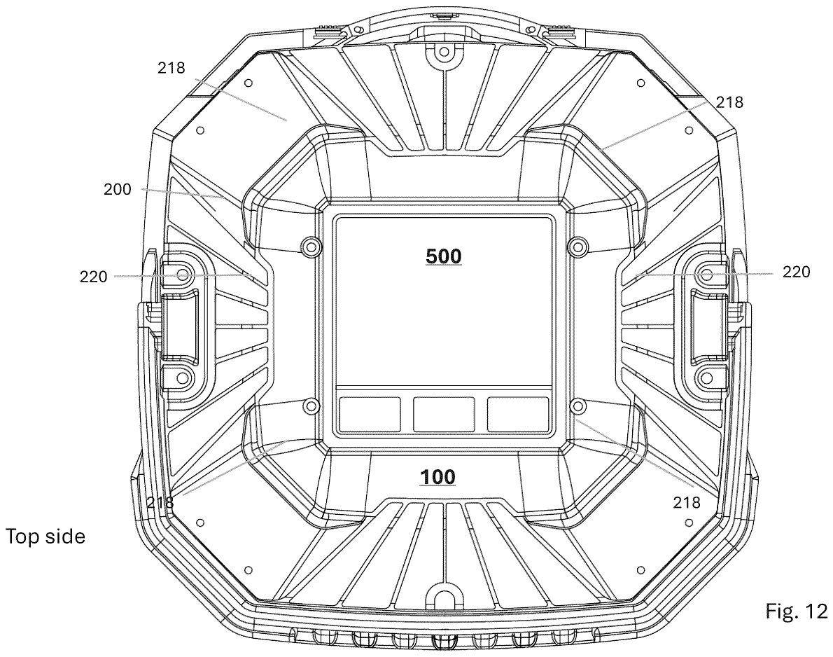

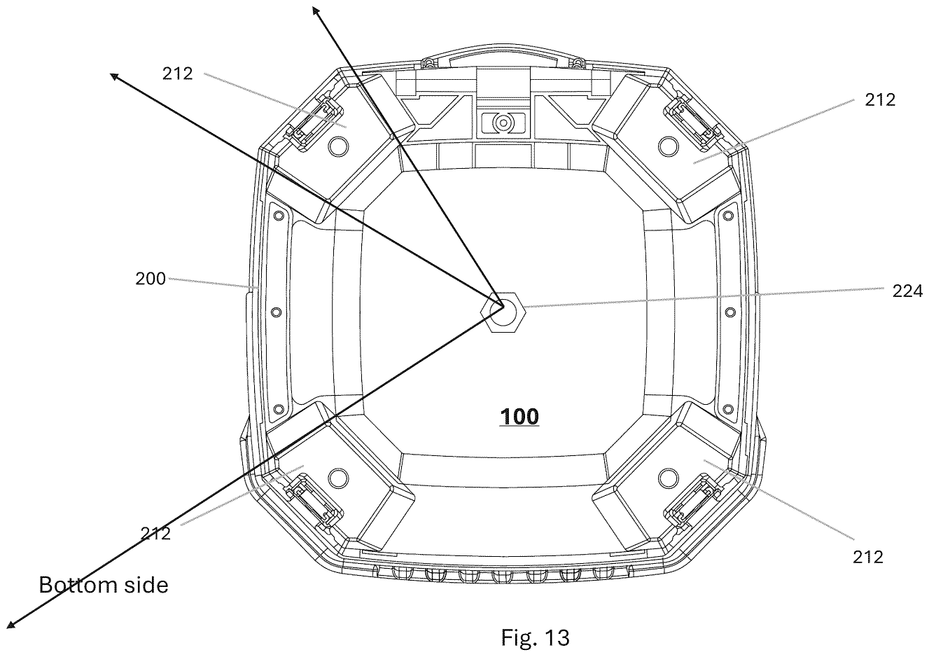

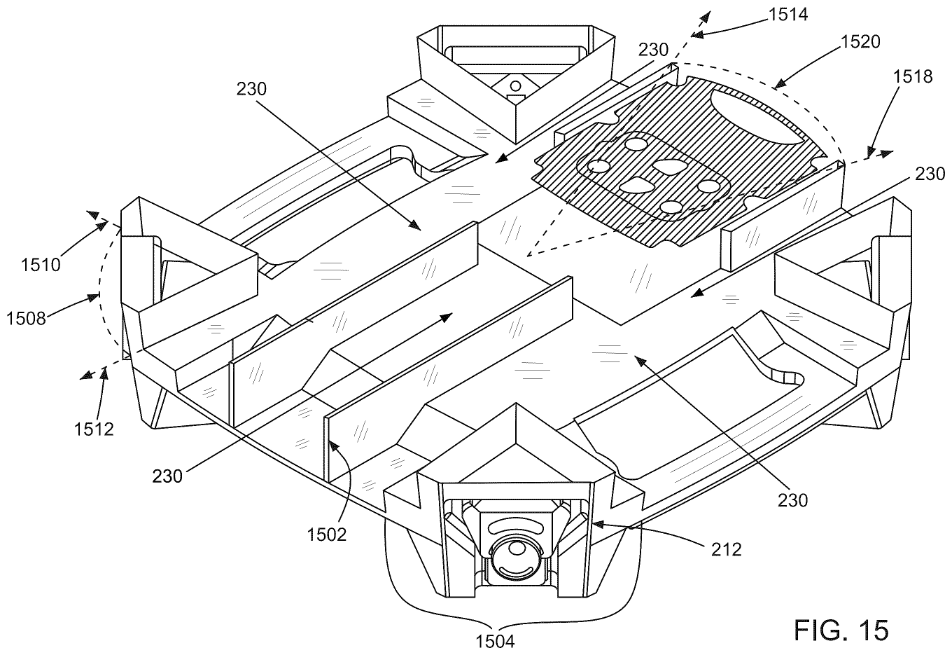

The area monitoring units 100 may be stacked. Stacking the area monitoring units 100 may assist in storage, transportation, and/or deployment of the area monitoring units at a work site. Stacking area monitoring units may facilitate easily deploying multiple sensors without having to modify any individual area monitoring unit. Instead, an existing monitoring unit including the desired sensor can simply be stacked with other area monitoring units to provide the desired sensing configuration for the area. For example, with reference to FIG. 12, which shows a top of an area monitoring unit 100 according to example embodiments, an area monitoring unit 100 may have a generally square shape (e.g., “squared off”) and be symmetrical such that, for example, it can be rotated 90, 180, 270 or 360 degrees, and still stack with area monitoring units 100 above and/or beneath it. For example, each side of the area monitoring unit 100, such as shown in FIGS. 8-11, may have a same width to provide a square shape for the area monitoring unit 100. This may provide an advantage of being able to easily stack the monitoring units 100 without the need to consider the rotation of each area monitoring unit 100 relative to each other. Additionally, in some examples, if the center of gravity of each of a number of area monitoring units 100 to be stacked is off-center, it may be desirable to have respective area monitoring units 100 rotated 180 degrees relative to each other (or, in another example, at progressive 90 degree turns from each other) as they are stacked to maintain a center of gravity at the center of the stack of area monitoring units 100. This may enhance the stability of the stacked area monitoring units 100 (e.g., such that they are less likely to tip over) and may provide for easier transport and carrying. Further, the stacking support aspects, including the configurable bottom portion (e.g., reference FIG. 13, FIG. 15, or element 180 FIG. 17), which may include channels to support sufficient gas flow through the monitoring device to a sensing element in the core (e.g., reference 250 in FIG. 11), and the upper face (e.g., reference 216 in FIG. 7), can be configured with matching stacking features to facilitate ease of stacking. In certain embodiments, the configurable bottom portion may be embodied as, and/or described as, a “tray” in the present disclosure. In certain embodiments, the bottom portion 180 may be integral with a main body of a monitor. In certain embodiments, the bottom portion 180 may omit stacking features (e.g., to provide a contact surface of the tray that conforms to a mounting surface), and/or the stacking features may be separable from the remainder of the bottom portion 180, allowing for separate contact surfaces of the bottom portion 180 for transport and mounting/deployment. In the example of FIG. 7, the upper face includes an engagement notch 218 that receives and engages a protrusion 212 integrated on the configurable bottom portion. The stacking features may include a locking mechanism 214 allowing for stacked monitors to be secured together, and separated, without requiring a tool or the like.

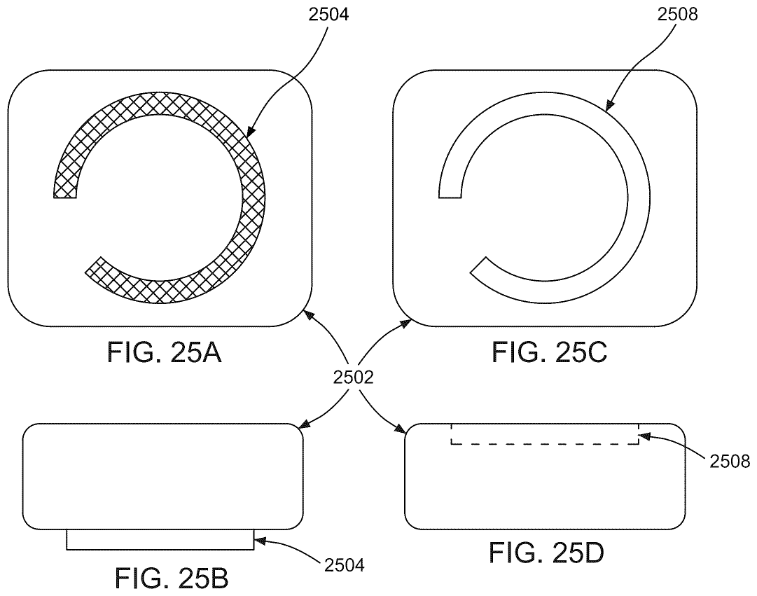

In the example of FIG. 25A-25D, the gas monitor 2502 includes a curved engagement protrusion 2504 on one surface, and a curved engagement slot 2508 on another end. FIG. 25A depicts a bottom-up view of the gas monitor 2502 having a curved engagement protrusion 2504 on a bottom surface. The curved engagement protrusion 2504 may be semi-circular, oval, circular, arced, or the like. FIG. 25B depicts the curved engagement protrusion 2504 and gas monitor 2502 in profile. FIG. 25C depicts a top-down view of the gas monitor 2502 having a curved engagement slot 2508 on its top surface while FIG. 25D depicts the curved engagement slot 2508 and gas monitor 2502 in profile. In this example, the gas monitors may stack by engaging the bottom of one gas monitor using the curved engagement protrusion 2504 into the top of another gas monitor's curved engagement slot 2508. In some examples, the curved engagement protrusion 2504 is on the top surface and the curved engagement slot 2508 is on the bottom surface. In this example, the curved engagement protrusion 2504 may not extend into or form part of a central region of air flow within the housing. In these examples, configurations of the gas monitor that have increased air flow accessibility are possible. The examples herein utilizing protrusions 212 and/or curved engagement protrusions 2504 are non-limiting examples, and embodiments herein utilizing stacking features may include any combination of protrusions and slots, including for example piece-wise protrusions, mixed protrusions and slots on the top and bottom surfaces, or the like. In certain embodiments, the protrusion and slot configuration can operate as a keying interface, enforcing proper orientation of the stacked gas monitors (for embodiments where the orientation of the stacked gas monitors is of interest).

In embodiments, the area monitoring unit 100 may be embodied in suitable form factors, such as any icosahedron, any 3-d prism, any shape where the stack of shapes keeps the center of mass defined within the support points, or any symmetrical shape. One of skill in the art, having the benefit of the disclosure herein, can readily determine how to choose disclosed form factors from the present disclosure to use for particular area monitoring units. Certain considerations for the person of skill in the art in determining the form factor to use include, without limitation: the type of installation, the location of installation, the number of units being deployed, the ease of manufacturing the unit, the arrangement of units, the size of units, or environmental considerations such as temperature, pressure, humidity, the size and shape of transport facilities (e.g., truck beds and/or boxes, cart platforms, etc.), and/or the planned number of units in a load to be handled by deployment personnel.

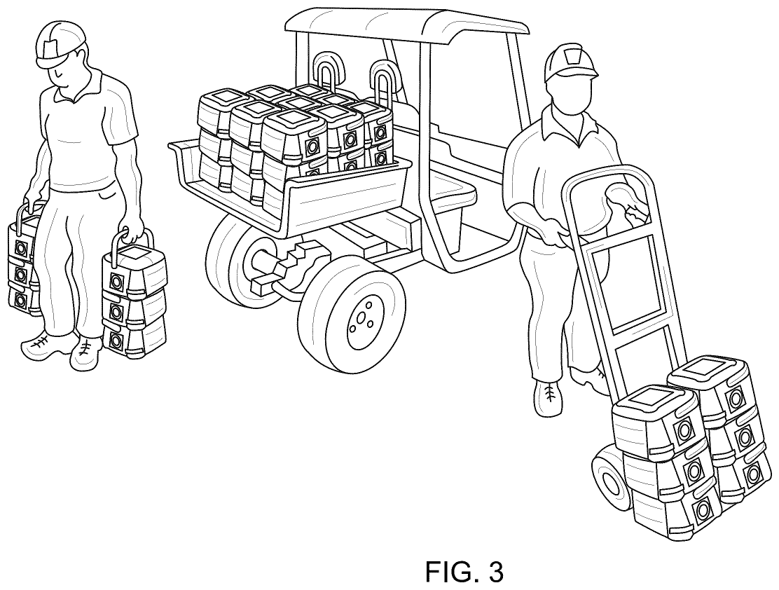

Certain deployments of monitors in an area require many monitors to be deployed, for example to support a fenceline perimeter, to ensure all confined spaces or other areas of interest are covered, and/or to ensure coverage for very large locations such as an oilfield, mine, or the like. An example deployment can include hundreds of monitors, often positioned by hand in the final stages, and aspects herein such as the stacking features, uniform shaping and predictable weight distribution to facilitate manual handling, and the integrated handle, improve the ease of handling, support for proper ergonomic form in handling, movement of monitors and/or stacks of monitors between transportation methods (e.g., shelf to truck to deployment vehicle), and recovery of monitors (e.g., at the end of a deployment). FIG. 3 depicts a deployment example, with typical non-limiting operations that may be performed during a deployment illustrated. The example monitors are depicted roughly to scale for a person according to an example embodiment.

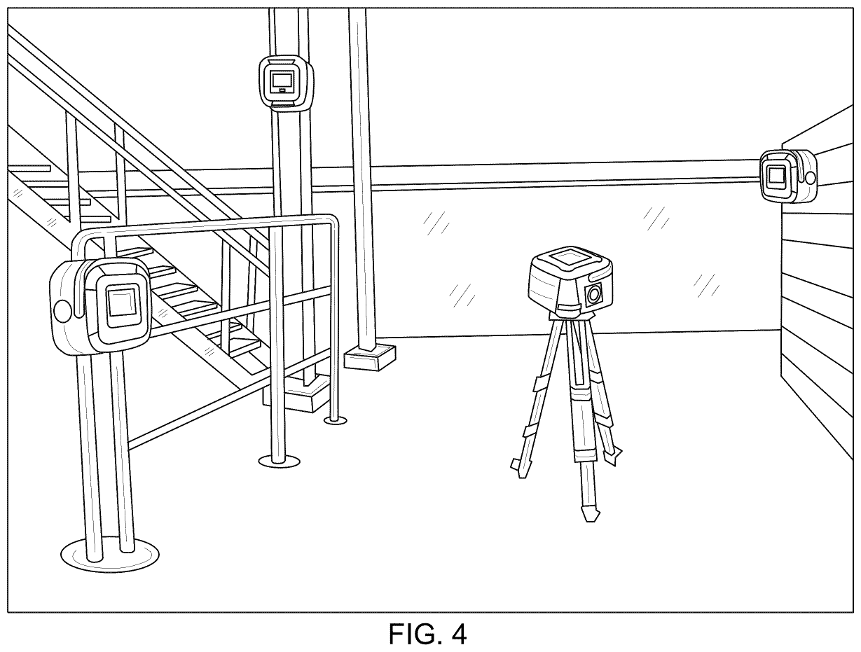

With reference to FIG. 12, in some example embodiments, the top of the area monitoring unit 100 may include four notches 218 at the four corners of housing 200 as well as a user interface 500. With reference to FIG. 13, which shows the bottom portion 180 of an example area monitoring unit 100 according to example embodiments, viewed from the bottom (e.g., the contact surface, for example in the mounting examples depicted in FIGS. 4-6). The example bottom portion 180 of an area monitoring unit 100 may include four protrusions (e.g., “feet: or “knobs”) 212 at the four corners of housing 200. These four feet 212 of an area monitoring unit 100 may be structured to interface with the four notches 210 of another area monitoring unit 100 stacked thereunder to secure the positioning of the area monitoring units 100 relative to each other. In certain embodiments, the notches/feet may be keyed, for example to ensure consistent orientation of the stacked monitors, and/or in certain embodiments the monitors may be stacked in any orientation. Orientation control of the monitors may provided in any manner—for example if the four possible orientations between two stacked monitors are labeled ABCD, where A in the example is full alignment (e.g., each corresponding corner in the upper monitor is in the same position as the corresponding corner in the lower monitor), and BCD are clockwise rotations from that alignment (e.g., the upper left corner of the upper monitor is the matching element of the monitor compared to the upper right corner of the lower monitor, in the “B” orientation), then it may be useful to allow one of the following orientation regimes (e.g., utilizing keying for hard enforcement, and/or labeling or alignment markers for soft enforcement and/or to facilitate ease of deployment operations), without limitation: A (e.g., enforcing full alignment); B (e.g., enforcing a rotation of each stacked monitor, for example to facilitate a center of mass trajectory for the stack); BD (e.g., enforcing a rotation of 90° or 270°, for example to facilitate a center of mass trajectory for the stack); and/or AC (e.g., enforcing alignment or 180° flipping, for example to facilitate a center of mass trajectory for the stack). Other alignment regimes are contemplated herein, and one of skill in the art, having the benefit of the present disclosure, can readily determine a configuration for the bottom portion 180 and the upper face that provides the selected orientation regime. Certain considerations for determining the alignment regime for a given embodiment include, without limitation: the distribution of mass within the monitoring unit; ease of operations for handlers in the deployment process; and/or the cost of manufacturing components of the monitoring unit to provide for a given orientation regime.

Additionally, as shown by example in FIG. 7, each foot 212 of an area monitoring unit 100 may include a locking mechanism 214 (which may include a latch) that engages with a corresponding notch 210 of another area monitoring unit 100 stacked thereunder, such as a structure within the notch 210 that engages the deployed latch. Thus, the area monitoring units 100 may be locked and secured together as they are stacked, forming one unit for transport.

As discussed elsewhere herein, the empty gas flow channels positioned low in the gas monitor results in a raised center of mass. The heavy battery pack(s) being positioned above this low mass area poses numerous challenges with respect to ease of stacking, ease of deployment, and ease of access for battery replacement, to name a few. Surprisingly, the inventors found that the addition of a support assembly and an engagement assembly, along with additional features (e.g., locking tab, keyed engagement), enabled stacking and deployment of the top-heavy gas monitors with ease while retaining access to the battery packs throughout storage, transport, and deployment. An example gas monitor may include a housing defining a sensor assembly. The housing may include a support assembly at a first end (such as protrusion 212), the support assembly comprising a plurality of extended members (such as engagement notch 218), and an engagement assembly at a second end, the engagement assembly comprising a plurality of receiving slots matched to engage the plurality of extended members. The engagement of the receiving slots of one gas monitor by the extended members of another gas monitor enables stacking the gas monitors and ensures that the gas monitors remain upright when stacked. Stacking is possible within certain tolerances of the engagement, that is, the engagement does not have to be positionally exact. For example, there may be an offset by ½ inch that would still allow for stacking. Additionally, displacement of the stacked monitors may be a design choice, for example to implement a slant in the monitor stack to accommodate certain shelf or transport options, to facilitate ease of reaching or removing monitors from the stack by deployment personnel, or the like. Any support assembly arrangement that allows for, or provides for, displacement between stacked monitors, while maintaining the center of mass of the stack (e.g., for at least a predetermined number of stacked monitors, such as but not limited to, up to six monitors in a stack) within a footprint of the extended members of the lowest gas monitor is contemplated herein. Position changes could be rotated, but could include horizontal displacement or tilting to accommodate certain mounting types or surfaces for placement. The shape of the monitor may enable, allow, or preclude positional exactness.

A geometry of the support assembly and the engagement assembly are configured such that a center of mass of two stacked gas monitors is positioned within a footprint of the plurality of extended members of the support assembly of a lower one of the two stacked gas monitors.

The geometry of the support assembly and the engagement assembly may be configured such that a center of mass of n (n is a number between 2 and 6, inclusive) stacked gas monitors is positioned within the footprint of the plurality of extended members of the support assembly of a bottom one of the n stacked gas monitors.

Support assembly shape could be defined by the actual positions of the extended members (e.g., a dot-to-dot), or by the housing frame where the extended members are attached. For example, a circular housing with four extended members could be described as having a circular support assembly and/or a square support assembly.

In some embodiments, the support assembly defines a regular polygon, and the plurality of extended members are positioned at vertices of the regular polygon. For example, the bottom side of the gas monitor depicted in FIG. 13 has protrusions 212 that define the polygon shape of the gas monitor. The regular polygon has between 3 and 12 sides, inclusive. In some embodiments, the support assembly defines a polygon (e.g., which may be a regular or irregular polygon), and the plurality of extended members are positioned at vertices of the polygon. For gas monitors with point-like extended members of the support assembly, an integer number of extended members defines a polygon. For gas monitors with more complex protrusions of the support assembly, for example with portions that are curved and/or have a significant linear extent, such protrusions may not define a polygon. In some embodiments, the housing defines a plurality of flow channels. A flow management portion, which is adjacent to the support assembly, includes a vertical extent of the plurality of flow channels. In some embodiments, the flow management portion includes an at least 20% azimuthally open configuration.

In certain embodiments, the gas monitor further includes a battery pack positioned vertically above the flow management portion.

In these embodiments, the sensor assembly may be configured to monitor between one and seven, or up to twelve, gases, inclusive. The gas monitor may include cellular connectivity, and the battery pack may be sized to provide normal operations for the gas monitor for at least 25 days. Gas sensing and cellular connectivity come at the expense of high-power consumption. Battery pack sizing affects the overall weight of the gas monitor while also impacting whether there is sufficient capacity for long-term, mounted and/or remote operations, and is therefore an important consideration for deployment. In some embodiments, the gas monitor may have a mass of less than eight kilograms, which facilitates ease of deployment.

In certain embodiments, the gas monitor may further include a locking tab, such as locking mechanism 214, configured to selectively couple at least two of the plurality of extended members to corresponding receiving slots.

In certain embodiments, the support assembly and the engagement assembly are keyed to enforce a selected orientation between the lower one of the two stacked gas monitors and an upper one of the two stacked gas monitors. Keying enforces any orientation. There may be advantages to rotating each stacked monitor by X degrees (e.g., 90°), especially where the mass is not uniform (e.g., the battery pack can make the mass asymmetrical). Keying allows orientation to be readily enforced without relying on the deployer, and easing the resource load on the deployer. Further, keying supports fully aligned stacking.

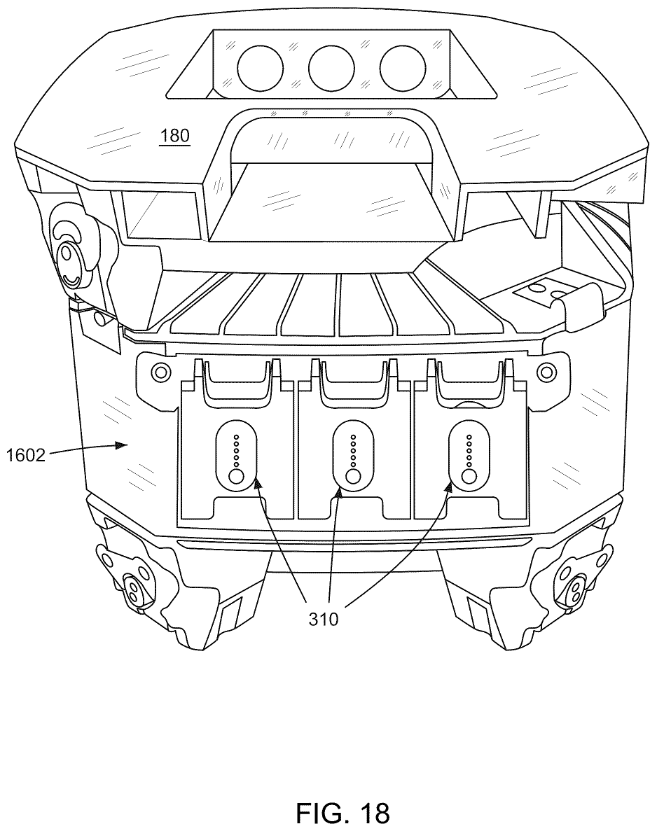

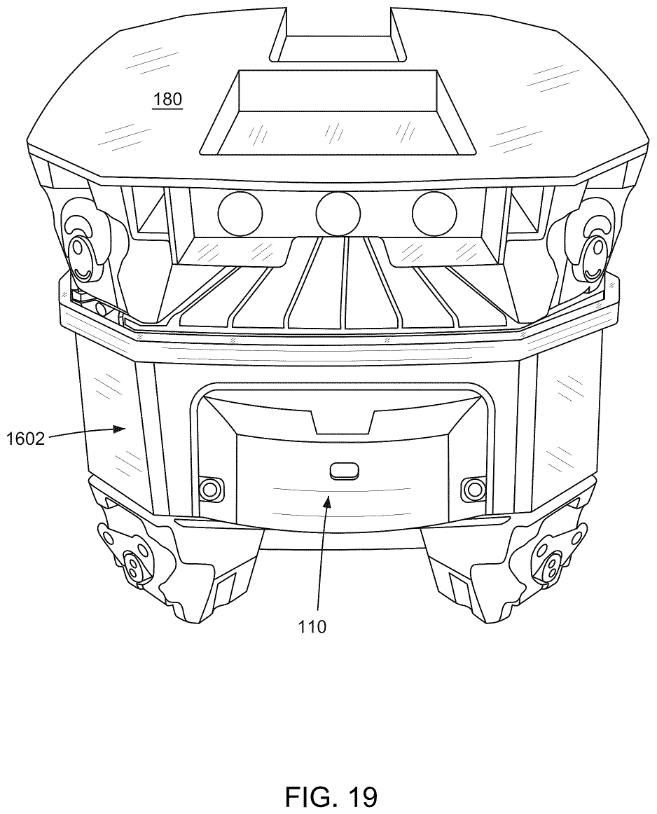

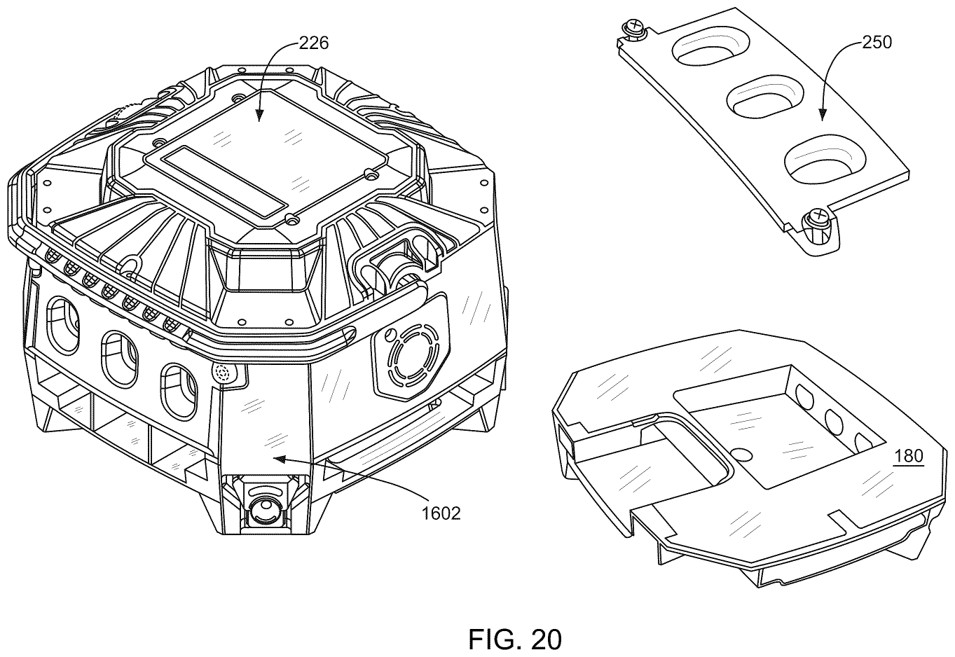

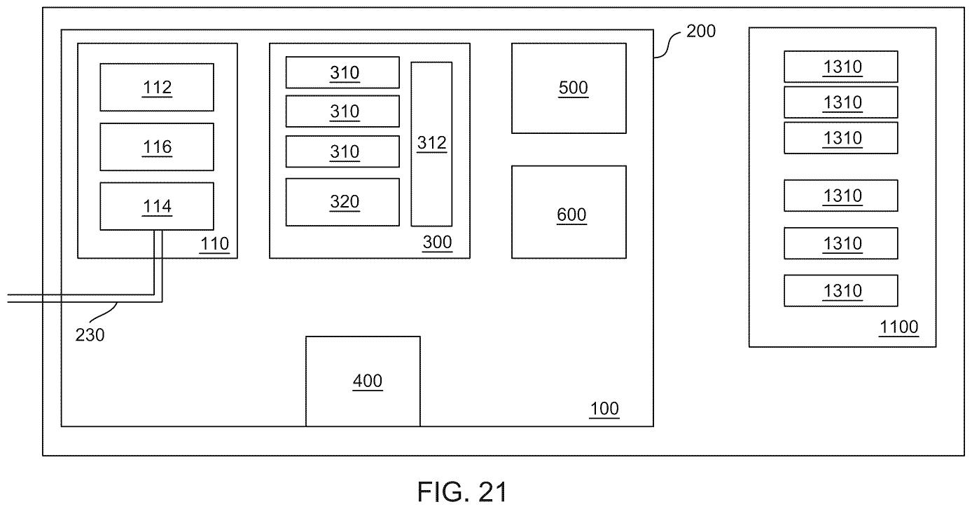

In certain embodiments, a battery pack 1100 (reference FIG. 21, depicted schematically) may be provided with the monitor 100, for example to provide additional power for long-term monitoring without requiring battery replacement during the deployment, and/or reducing the number of battery replacement and/or recharge operations that need to be performed to support the deployment. In certain embodiments, separate battery packs 1100 may be stacked and locked together with the area monitoring units 100. For example, the battery packs 1100 may have the same top and bottom footprint (at least with regard to notches 218 and/or feet 212) as an area monitoring unit, even though a thickness of a battery pack may not be the same (e.g., typically the battery pack will be thinner, but may be configured to be the same and/or may be greater) that of an area monitoring unit 100. Additionally, the battery packs 1100 may have a locking mechanism like the locking mechanism 214 to engage with a corresponding notch underneath, and the notches of the battery packs 1100 may be structured to engage with the locking mechanism of another battery pack and/or an area monitoring unit 100. Therefore, in example embodiments, a plurality of area monitoring units and/or battery packs 1110 may be stacked and locked together to form one single, transportable unit.

In example embodiments, an area monitoring unit 100 may include a ring or other attachment mechanism, such as a D-ring or hook, on its top side (e.g., the top side shown in FIG. 12). Thus, the attachment mechanism of an area monitoring unit 100, or, for example, the top area monitoring unit 100 of a plurality of stacked area monitor units 100, may be used to hoist the stacked units 100/1100 using, for example, using a pulley or lift system. This may avoid the need to hand-carry the units 100 up stairs or a ladder of a workplace, such as a tower. The example of FIG. 12 does not depict a D-ring or hook. In certain embodiments, a handle 222 may be mounted to the top side, facilitating ease of carrying while still allowing for stacking and deployment with the handle in position—for example with a handle that rotates between an extended and a stored position.

With reference to FIGS. 7-13, in example embodiments, the area monitoring unit 100 may provide a form factor that is more compact than devices of the related art. Owing to its compact size and configurable contact surface of the bottom portion 180, the area monitoring unit 100 may be suitable for mounting to various structures, such as walls, rails, scaffolding, beams, columns, etc., through various mounting capabilities. For example, in some embodiments, an area monitoring unit 100 may be provided with a magnet, an adapter plate for wall or other mounting, etc. In some embodiments, with reference to FIG. 13, an area monitoring unit 100 may include, for example, a universal tripod connector 224 for mounting the area monitoring unit 100 on a tripod, e.g., via a bolt in the tripod connecting to the universal tripod connector 220.

In example embodiments, the area monitoring unit 100 may be made more or less suitable for a public location, such as, for example, through a selected color of its housing 200 (e.g., to render the monitor more or less visible, and/or to match a color scheme that may be selected for reasons of safety, logistics, and/or aesthetics), the use of a face plate(s) 226 (e.g., reference FIG. 20) to cover certain electric components, connections 400, the user interface 500 and/or other displays, for example to prevent interference with and/or reading of the user interface 500. For example, the area monitoring unit 100 may be mounted at a public event, such as a parade, with its user interface 500 covered by a face plate 226, but may nevertheless have the ability to sound and/or trigger alerts without the public being able to read notifications on the user interface or interfere therewith.

With reference to FIG. 21, an area monitoring unit 100 according to example embodiments may include, within the housing 200, a core module 110 including at least one processor 112, at least one sensing module 114, and at least one memory 116, which, in some embodiments, may include separate circuitry and/or printed circuit boards. In example embodiments, memory 116 may include one or more non-transitory computer-readable storage mediums, and may store instructions for execution by the processor 112. As will be apparent to one skilled in the art upon reading this disclosure, these instructions, together with their execution via processor 112, may provide aspects of the example embodiments as described herein. The example sensor may be a sensor of any type, including one or more gas monitoring sensors, but may additionally or alternatively include any sensing capabilities desired to support the monitoring functions, such as and without limitation, temperature sensing, pressure sensing, humidity sensing, noise detection, and/or vibration detection.

The area monitoring unit 100 may include a gas path 230, which may be described in greater detail with reference to FIG. 15, and which may provide gas from at least three directions (e.g., at least three orthogonal planes) to the at least one sensing module 114 such that the area monitoring unit 100 may be positioned in either a horizontal or vertical orientation and still provide proper gas flow to the sensing module 114. The utilization of a gas path 230, which may be defined by one or more gas flow management portions 1502, allows for the monitor to be mounted in multiple orientations or rotations, and to ensure proper gas flow in relatively confined spaces (the air surrounding the monitor should still be representative of the air that is being monitored). The at least one sensing module 114 may, for example, include one or more gas sensors, including, for example, a hazardous gas sensor. In an example, the sensing module 114 may be structured to detect up to twelve gases simultaneously. In some embodiments, the protrusion 212 that supports some orientations and/or placements of the gas monitor may also include or form part of a flow management portion 1502.

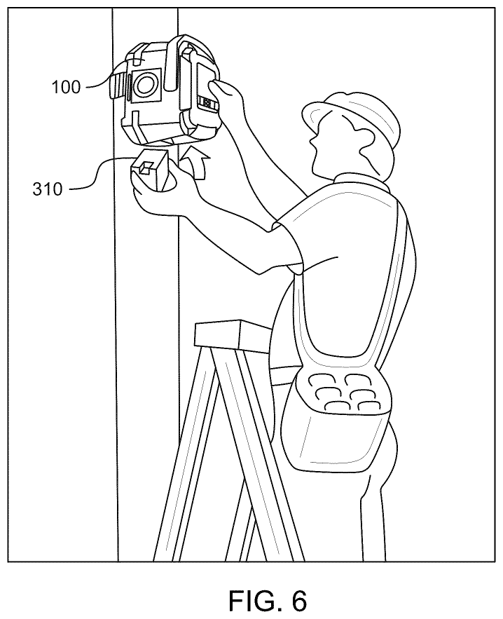

The area monitoring unit 100 according to example embodiments may include a power supply 300, also known herein as a battery assembly, including one or more batteries 310. For example, some embodiments may include at least three batteries 310. The power supply 300 may include circuitry 320 (e.g., electronic circuitry), which may include charging circuitry to charge the batteries 310 from, e.g., power from a solar panel as described herein (e.g., as received via the at least one connector 400) to charge the batteries 310. The circuitry 320 may include hot swap circuitry to continue powering the area monitoring unit 100 via one or more of the batteries 310 while the other battery/batteries 310 are being replaced. For example, FIG. 6 illustrates a user replacing a battery 310 in an area monitoring device 100.

With reference again to FIG. 21, the power supply 300 may include an interconnect board 312 to which each of the batteries 310 is connected, and which may provide power transfer between the batteries 310, the circuitry 320, and the remainder of the area monitoring unit 100. Each of the batteries 310 may include blades that contact the interconnect board 312—in the example of FIG. 18 the blades are on the side inserted into the monitor and not depicted. In an example, the blades may be molded to help with an Ingress Protection (IP) Seal. In certain embodiments, the battery connection to the board may be provided as a spark proof or explosion proof connection, and/or the connection may be configured to meet any selected industrial standard(s), for example according to the monitoring environment.

The housing 200 of the area monitoring unit may include at least one connector 400 for, e.g., connecting to a solar panel or battery pack 1100, as discussed in more detail herein.

The area monitoring unit 100 may include a user interface 500 for interacting with one or more users. Additionally, the area monitoring unit 100 may include other interfaces 600, which may include, for example, a wireless module (e.g., WiFi), a Bluetooth module, an NFC interface, another peer-to-peer wireless interface, a proprietary wireless interface, a cellular module, a satellite transceiver module, an Ethernet module, etc., for, e.g., communicating with users, other area monitoring units 100 or sensing devices, a system such as a cloud server, other Internet locations, etc.

With reference to FIG. 21, example embodiments may include a battery pack 1100 having a plurality of batteries 1310. For example, the battery pack 1100 may include six batteries 1310, and/or otherwise have twice the designed power storage capacity as the batteries 310 of power supply 300 internal to the area monitoring unit 100. In some embodiments, the batteries 1310 may correspond to (e.g., be identical in structure and/or function) to batteries 310. As described herein, the battery pack 1100 may connect with and stack with area monitoring units 100. The number and type of batteries in the battery pack 1100, as well as the total energy storage of the battery pack 1100, is selectable according to considerations of the planned deployment. The utilization of an add-on battery pack 1100 is optional and non-limiting.

In example embodiments, the core module 110 may include an identification (e.g., such as a number or other alphanumeric string of characters), which may, for example, be stored in memory 116. Within the core, the sensing module 114, and/or each individual sensor within sensing module 114, may also include an identification, which may be stored in memory 116, and/or in a memory local to the sensing module 114 and/or each individual sensor therein. Likewise, the power supply 300 and/or each battery 310 (as well as each battery 1310) may include an identification, which may be stored in a memory local to the power supply 300 and/or each battery 300. These identifications, which may be unique to each component therewith, may be used for identifying themselves to, e.g., a remote system such as a central cloud server, gateway, or other area monitoring units 100. The core module 110 may also store in memory 116 a calibration history of the sensor(s) of sensing module 114.

An example gas monitor includes a housing defining a sensor assembly 114, a cellular capable transceiver 600, and a battery assembly 300 comprising a plurality of slots (e.g., three slots) configured to each receive a battery pack 310, and wherein the gas monitor is configured to operate normally with power provided by a single battery pack. While the example gas monitor can be configured with multiple battery packs and can operate with multiple battery packs, it can also operate off of just a single battery pack. To facilitate this capability, the battery packs may be coupled in parallel rather than series, however, other technologies, such as solid state voltage conversion, may also facilitate this capability. Arranged as it is to receive multiple battery packs, the example gas monitor is remarkable in its capacity to operate in high power consumption modes, such as in operating wirelessly, operating for long durations, or in powering multiple functions/sensors of the monitor. The gas monitor is configured to continue normal operation in response to a removal of a battery pack.

Some deployed configurations pose major physical challenges, however, the inventors have devised a way to meet the challenge of airflow, high power consumption, and remote communication, while retaining the ease of battery replacement, stacking, transport, and mounting. While the remarkable configuration of being able to have a plurality of battery packs onboard the gas monitor extends the interval between battery replacements, when battery replacement is required, the battery assembly is configured to be accessible when the gas monitor is in a deployed configuration, and to continue monitoring and communications during the battery swap, reducing downtime and avoiding undesirable consequences for complete loss of power, such as rebooting, resetting or loss of volatile data, or the like, which is notable given the deployment challenges described herein. For example, the desire to maximize airflow through multiple flow channels results in a raised center of mass, and the desire to dispose multiple battery packs onboard results in portions of the gas monitor being disproportionately weighted. Given these challenges, to still be able to readily access the battery assembly while the gas monitor is deployed and operational/in service is unexpected. A field swappable battery with multiple battery options reduces downtime while increasing the runtime and reducing the number of trips required into the field to maintain one or more monitors.

In some embodiments, at least one of the plurality of slots is configured to couple to an external power source. For example, where there are three slots, there may be two batteries to swap, plus a slot for external power (e.g., an external battery pack or solar power source).

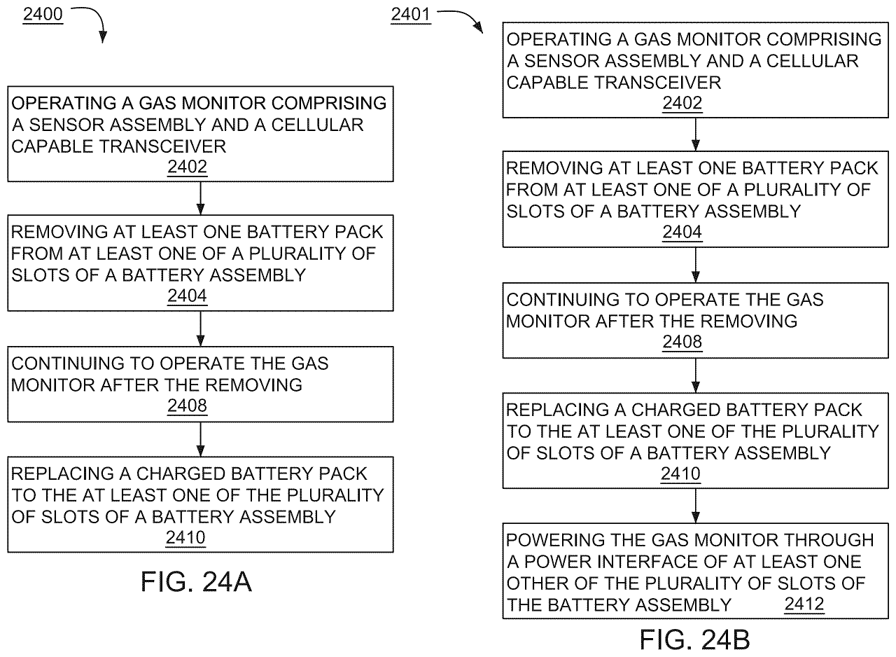

Referring to FIG. 24A, an example method 2400 may include a step 2402 of operating a gas monitor comprising a sensor assembly and a cellular capable transceiver. The example method may include a step 2404 of removing at least one battery pack from at least one of a plurality of slots of a battery assembly. The example method may include a step 2408 of continuing to operate the gas monitor after the removing. The example method may include a step 2410 of replacing a charged battery pack to the at least one of the plurality of slots of the battery assembly.

Referring to FIG. 24B, in an example method 2401, the step 2408 of continuing to operate may include a step 2412 of powering the gas monitor through a power interface of at least one other of the plurality of slots of the battery assembly.

In the example methods 2400, 2401, powering the gas monitor through the power interface includes powering the gas monitor with another battery pack or an external power source (e.g., an external battery pack, a solar power source).

In example embodiments, the core module 110 may be swappable from the area monitoring unit 100. For example, a faceplate 226 may be removed from the housing 200 of the area monitoring unit 100, and the core module 110 may be accessed and removed through the removal of one or more screws or other fastening devices. The core module 110 can thereby be replaced with a different core module 110 (e.g., one having a different sensing module 114 with different sensors therein), and/or added to a different area monitoring unit 100.

Because the core module 110 retains with it its identification stored in memory 116, the core module 110 may be tracked (including utilization and other data) regardless of which area monitoring unit 100 it is installed in. Likewise, the calibration history of sensing module 114, which may be included in the core module 110, may be retained and tracked so that an accurate knowledge of the calibration history may be maintained even if the core module 110 is swapped into a different area monitoring unit 100.

Similarly, because the batteries 310 may each retain with them their respective identifications, such batteries 310 may be tracked regardless of which area monitoring unit 100 they are installed in. Thus, for example, a detailed utilization history of each battery 310 may be maintained by a remote system, such as a cloud server or other centralized database, and the system may thereby know when a battery is in need of replacement or other maintenance.

With reference to FIG. 21, the area monitoring unit 100 may include a plurality of batteries 310. As described herein, these batteries 310 may be part of a power supply 300, although embodiments are not limited thereto. The batteries 310 may be connected in parallel. The batteries 310 may, for example, be 5 volt batteries. In an example, there are three batteries 310. By having three batteries 310, the area monitoring unit 100 may triple the runtime of a unit having only one battery 310, lengthening the time needed between maintenance. Each of the batteries 310 may be individually replaceable. Because each of the batteries 310 can be hot swapped, e.g., by operation of the hot swap circuitry, the area monitoring unit 100 may not need to be powered off when one or more of the batteries 310 is replaced, and/or all of the batteries 310 may be replaced sequentially. For example, the area monitoring unit 100 may be powered by any one of the batteries 310, as one, multiple, or all of the remainder of the batteries 310 are replaced. Additionally, in some embodiments, a solar panel may provide sufficient power to the power supply 300 of the area monitoring unit 100—for example, via a connection 400—such that all of the batteries 310 may be replaced (e.g., taken out at the same time) and the power supply 300 may continue to power the area monitoring unit 100 during such replacement. Additionally, because the batteries 310 are easily replaceable, there may be no need or desire to run independent charging cables from outlets to each area monitoring unit 100. Additional connections 400 to a solar panel, an additional battery pack, and/or an external power source may be provided as desired, and such connections may be configured to be spark proof, explosion proof, and/or according to a selected industrial standard.

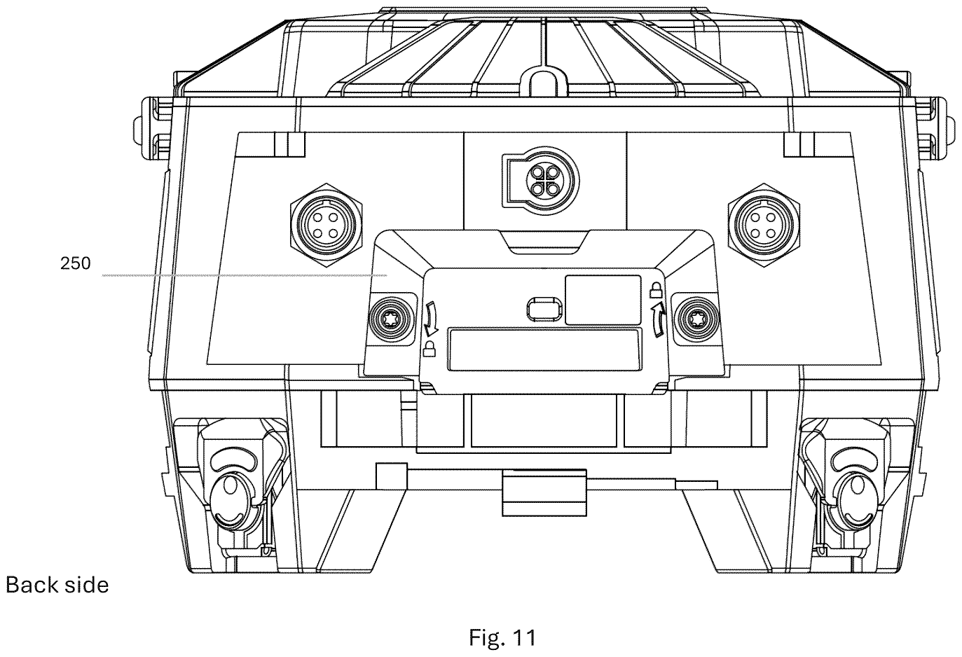

In example embodiments, with reference to FIGS. 7 and 10, to replace the batteries 310, a face plate 250 on the housing 200 may be removed via a plurality of screws or other attachment mechanisms (for example, two screws), providing access to the batteries 310 therein and satisfying certification requirements for battery replacement in hazardous locations.

Being able to replace the batteries 310 of an area monitoring unit 100 without powering off the area monitoring unit 100 may provide a significant improvement to the user experience and overall operational efficiency for the deployment. For example, when an area monitoring unit 100 is powered off, policies enforced by regulation and/or private entities (e.g., a corporate policy) may require that tests be performed when it is powered back on—for example, checking sensor sensitivity (e.g., gas sensor sensitivity) for sensors of the sensing module 114, checking connectivity between modules to ensure functional operation, checking connectivity to a remote system such as a cloud server or other monitoring units, etc. By maintaining power to the area monitoring unit 100 while the batteries 310 are replaced, these tests may not be necessary and the battery replacement process may be expedited, resulting in significant time savings and reduced operational complexity for a large deployment.

With reference to FIG. 15, the area monitoring unit 100 may include a gas path 230, which may provide gas from multiple directions to the at least one sensing module 114. For example, the gas path 230 may be open to all sides (e.g., all four horizontal sides) of an area monitoring unit 100, and may provide flow paths for gas therefrom—e.g., as integrated into the housing 200—to the sensor(s) of sensing module 114. In certain embodiments, the gas path 230 may be provided from at least two sides, which may be opposing sides or adjacent sides, which still supports a range of mounting options. Thus, regardless of an area monitoring unit's 100 position, orientation, or objects immediately adjacent thereto, gas in the environment may still reach the sensing module 114 as long as at least one side of the housing 200 (and/or one side having a gas path 230 opening) is open to the air. The gas path 230 may allow gas from the external environment (e.g., a representative sample of the air) to come into direct contact with the sensor(s) of sensing module 114. The openings of the gas path 230 on each side of the area monitoring unit 100 may run in at least three directions (for example, an X, a Y, and a Z direction, which may be orthogonal) within the housing, such as to within a central region of the housing, to provide the external air to the sensor(s) of sensing module 114.

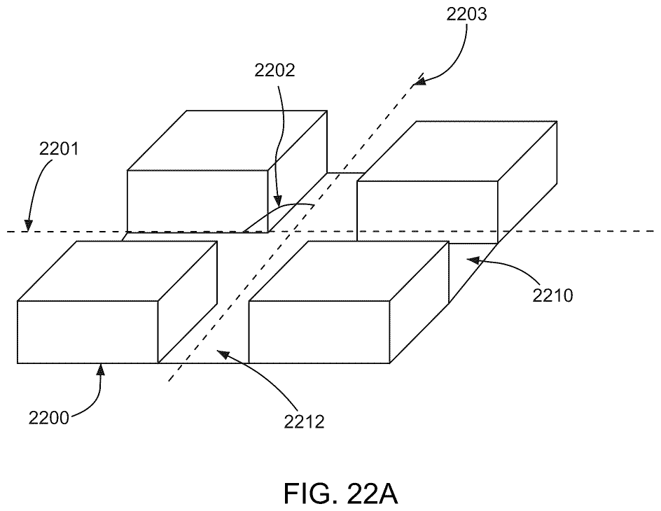

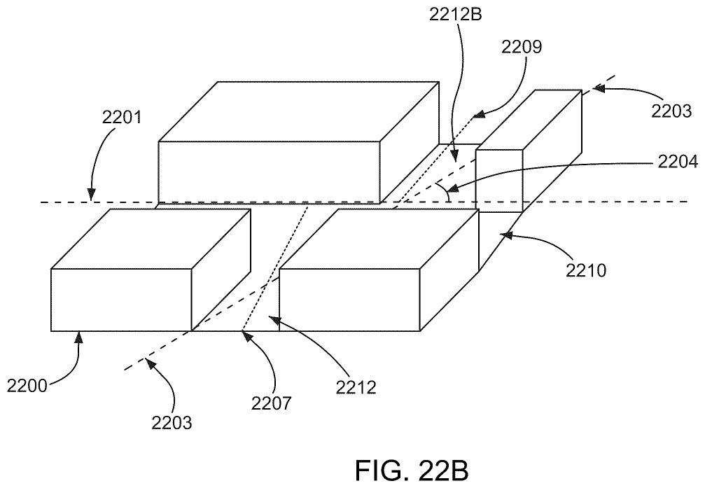

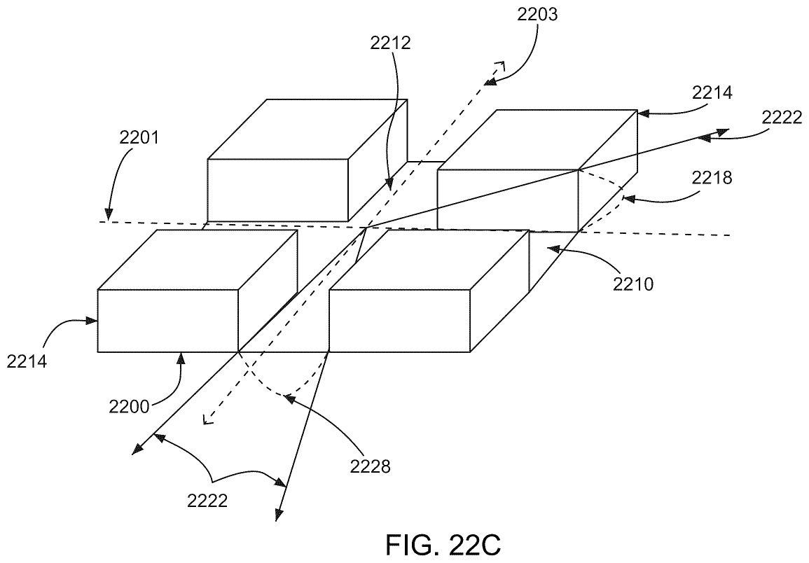

Referring now to FIG. 22A-22B, gas flow management in a gas monitor is disclosed. Throughout this disclosure, the area monitoring unit may be referred to herein interchangeably with terms such as gas monitor, area monitor, and monitor. An example gas monitor includes a housing defining a first flow channel and a second flow channel. A simplified schematic of a bottom portion 2200 of a gas monitor housing is depicted. In some embodiments of the housing, the first flow channel and the second flow channel define gas flow paths that are at a substantially 90 degree angle 2202 (FIG. 22A). It should be understood throughout this disclosure that the term substantially refers to the qualitative condition of exhibiting total or near-total extent, degree, or precision of a characteristic or property of interest. One of ordinary skill in the art will understand that certain measurements in the arts are often reported within a margin of error or described as being within tolerance. The term “substantially” is therefore used herein to capture characteristics and values with margins of error, variability, and/or within tolerance(s), and is intended to account for variability in manufacturing, assembly, and/or component accommodation within the housing. For example, for embodiments of gas paths disclosed as being disposed substantially at 90 degrees with respect to one another in the housing, the actual angle may be off by 5° or less. In another example, for embodiments of gas paths disclosed as being substantially perpendicular with respect to each other, the actual arrangement may be slightly off perpendicular. It should be understood that, in the case of gas flow paths, they may be designed to be operable within margins of error, variability, and/or within tolerance(s), such as to accommodate variability in area monitoring unit deployment, placement, and stacking arrangements.

Continuing with reference to FIG. 22A, the first flow channel 2210 includes a first flow path axis 2201 and the second flow channel 2212 includes a second flow path axis 2203. Referring now to FIG. 22B, in the example gas monitor, the first flow path axis 2201 intersects the second flow path axis 2203 at an angle of at least 60 degrees. For example, two gas paths separated at a 60 degree angle 2204 may mean that no incident wind can be more than 60 degrees out of the line of flow (against the maximum 120 degree gap between flow paths), and that there is still about 50% of maximum gas flow at any angle. In the example of FIG. 22B, gas flow path 2203 is depicted as traveling in a continuous line, however, given the offset structure of this bottom portion, gas flow channels that intersect 2210 may actually be considered to be two channels, channel 2212 and channel 2212B. Indeed, a gas path 2207 flowing through channel 2212 may intersect gas flow path 2201 at one location and a gas path 2209 flowing through channel 2212B may intersect gas flow path 2201 at another location.

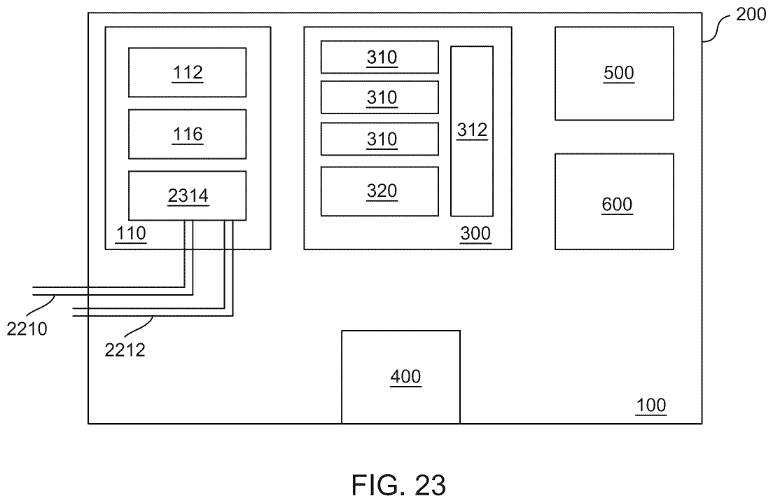

Referring to FIG. 23, a schematic of the gas monitor is depicted wherein a gas sensing element 2314 may be fluidly coupled to both of a first flow channel 2210 and a second flow channel 2212.

In some embodiments of the gas monitor, the first flow path axis 2201 intersects the second flow path axis 2203 at a substantially perpendicular angle, such as depicted in FIG. 22A.

In some embodiments, the housing comprises a flow management portion 2214 comprising a vertical extent of the first flow channel 2210 and the second flow channel 2212.

The flow management portion 2214 includes an at least 20% azimuthally open configuration. Having azimuthally open configurations in a gas monitor has numerous benefits, including ability to deploy the monitors in challenging locations where portions of the gas monitor may be obstructed, ability to measure gasses in environments where there may be low flow such as in an indoor deployment, and facilitating mounting of the gas monitor due to the placement of the open portions with respect to the remainder of the gas monitor.

The azimuth 2218 may be measured between a line-of-sight 2222 from the center of the bottom portion 2200 to outside the gas monitor and the bottom portion (or a line-of-sight 2222 defined by the bottom portion). The azimuth 2218 effectively defines a percentage of a circular arc that can be seen from outside the gas monitor. The center of the circular arc is positioned at the center of the bottom portion 2200. The azimuthally open configuration may include one or more azimuths 2218 associated with portions of the first flow channel 2210 and the second flow channel 2212 to total up to a percentage open configuration. In some embodiments, the flow management portion 2214 comprises an at least 50% azimuthally open configuration.

In some embodiments, the housing comprises a flow management portion 2214 comprising a horizontal extent of the first flow channel 2210 and the second flow channel 2212. In this embodiment, the azimuth 2228 may be measured between two lines-of-sight 2222 from the center of the bottom portion 2200 to outside the gas monitor. The azimuth 2228 effectively defines a percentage of a circular arc that can be seen from outside the gas monitor. The azimuthally open configuration may include one or more azimuths 2228 associated with portions of the first flow channel 2210 and the second flow channel 2212 to total up to a percentage open configuration. The one or more azimuths 2228 may define a percentage of the gas monitor perimeter that is open for flow, which may include curved flow paths.

In some embodiments, the gas monitor may include a mounting assembly at a first end of the gas monitor, and a display screen at an opposing second end of the gas monitor. This configuration may benefit deployment due to the ease of mounting in a configuration where the display panel is visible. The flow management portion 2214 may be adjacent to the mounting assembly, which results in the center of mass being raised in the gas monitor due to the low mass region of the flow channels being positioned low in the gas monitor. Raising the center of mass to include flow channels with enhanced azimuthally open configurations is a challenge with respect to efficient stacking, deployment, and transport of the gas monitors—a challenge that was impressively overcome by the inventors as described herein.

An example gas monitor includes a housing defining a plurality of flow channels, and a gas sensing element fluidly coupled to each of the plurality of flow channels at an intersection. An intersection may be where flow channels separate or diverge. An intersection may be where flow channels merge, converge, overlap, or join. An intersection may be where flow channels cross. In some embodiments, the flow channels include a ‘Y’ configuration. It should be understood that gas monitors may include more than one type of intersection. Each of the plurality of flow channels includes an average flow path trajectory. The average flow path trajectories at the intersection may be sequentially rotated by an angle between θ and φ inclusive, wherein θ comprises a value of 180/n, and wherein φ comprises a value of 120/n, wherein n comprises the number of the plurality of flow channels. For example, where there are four flow channels present, the sequential rotations would be the angle between the first and second channels (first sequential rotation), between the second and third channels (second sequential rotation), and between the third and fourth channels (third sequential rotation). In the example of FIG. 22C, the first flow channel 2210 and the second flow channel 2212 intersect at the center of the bottom portion 2200. In this example, n=2, so the average flow path trajectories at the intersection are sequentially rotated by an angle between 90° (θ) and 60° (φ) inclusive.

In some embodiments, the intersection includes a co-extensive, or overlapping, portion of the plurality of flow channels. In some embodiments, the intersection includes a co-extensive portion of the average flow path trajectories of the plurality of flow channels.

In some embodiments, the intersection includes a separating portion of at least a portion of the plurality of flow channels. For example, some channels may potentially have a shared portion (e.g., a Y configuration for two channels), and the intersection is where they separate.

In the example gas monitor, the housing includes a flow management portion comprising a vertical extent of the plurality of flow channels. The flow management portion includes an at least 20% azimuthally open configuration, an at least 30% azimuthally open configuration, at least 40% azimuthally open configuration, an at least 50% azimuthally open configuration, or an at least 60% azimuthally open configuration. The gas monitor may include a mounting assembly at a first end of the gas monitor, and a display screen at an opposing second end of the gas monitor, and the flow management portion may be adjacent to the mounting assembly.

In an example gas monitor, the housing defines a flow management portion 2214 that fluidly couples a central region of the gas monitor to ambient air. The central region is the space, or a portion thereof, defined in part by the flow management portion 2214 as well as one or more surfaces of the bottom portion 180 and/or surfaces of the gas monitor that abut the bottom portion 180. A gas sensing element (not shown) is fluidly coupled to the central region. For example, in FIG. 22C, the central region can be considered to be the entire space of the flow paths 2212, 2210 with a height defined by the flow management portion 2214 and the boundary defined by the edges of the bottom portion 2200. The flow management portion of the example gas monitor includes an at least 20% azimuthally open configuration, an at least 30% azimuthally open configuration, at least 40% azimuthally open configuration, an at least 50% azimuthally open configuration, an at least 60% azimuthally open configuration, or an at least 75% azimuthally open configuration. A skilled artisan would understand that the percentage of azimuthally open configuration in a gas monitor is impacted by support structures of the bottom portion 180, such as the flow management portion 2214 or 1502 and/or embodiments of the protrusion 212 that include flow management portions. For example, minimizing or modifying the size, shape, placement, texture, or orientation of the support structures may impact options for deployment of the gas monitor while at the same altering the percentage of azimuthally open configuration. For example, for a gas monitor that will be deployed mounted on a support beam for 100% of its operation may require only minimal support structures, and therefore may have azimuthally open configurations that may be greater than 60% and in some cases approaching 90%, while a gas monitor that is intended to travel to locations, such as with remote workers, may require robust support structures to enable flexible deployment options and may only support a 20% azimuthally open configuration. In the example of FIG. 25, where the curved engagement protrusion 2504 does not pass through the flow region of the gas monitor, as the protrusion 212 of FIG. 15 does, increased azimuthally open configurations may be possible.

The flow management portion of the example gas monitor may include one or more flow restrictions. For example, the flow management portion may include at least one flow restriction including an azimuthal arc of not greater than 25 degrees. FIG. 15 depicts a flow restriction 1504, which is the closed portion of the outer housing perimeter that does not allow flow. In the example of FIG. 15, there are four flow restrictions 1504, each defining a closed portion of the perimeter of the monitor, wherein each of the flow restrictions 1504 are defined by the size and shape of the protrusions 212. The azimuthal arc can be measured as the arc 1508 between two azimuths 1510, 1512 emanating from within the gas monitor and defining the outer edges of the flow restriction 1504. In the example of FIG. 15, there are four azimuthal arcs 1508 associated with the flow restrictions defined by the four protrusions 212 and each of the four azimuthal arcs 1508 are not greater than 25 degrees. In another example, where there is at least one flow restriction, the azimuthal arc is not greater than 120 degrees, which may result in or define a near 50% flow restriction.

In some embodiments, the at least one flow restriction includes three flow restrictions, each comprising an azimuthal arc of not greater than 30 degrees. In some embodiments, the at least one flow restriction includes four flow restrictions, each comprising an azimuthal arc of not greater than 25 degrees. In some embodiments, the at least one flow restriction includes five flow restrictions, wherein four of the flow restrictions each include an azimuthal arc of not greater than 25 degrees. In this embodiment, one of the five flow restrictions is the core module 110 (not shown in place). Azimuths 1514, 1518 define the azimuthal arc 1520 around the flow restriction of the core module 110, which is greater than 25 degrees. In this embodiment, the housing defines a plurality of external sides (e.g., four external sides), wherein the flow management portion includes open flow areas on each of the plurality of external sides. In the examples described herein, the gas monitor 100 is depicted with four sides, however it should be understood that it could be any number of sides. For example, the shape could be hexagonal, octagonal, triangular, or pentagonal. As shown in FIG. 15, each of the sides has a gas path 230 defining an open flow area.

In the example gas monitor, a mounting assembly is included at a first end of the gas monitor, and a display screen at an opposing second end of the gas monitor. This configuration may benefit deployment due to the ease of mounting in a configuration where the display panel is visible. The flow management portion 1504 may be adjacent to the mounting assembly, which results in the center of mass being raised in the gas monitor due to the low mass region of the flow channels being positioned low in the gas monitor. Raising the center of mass to include flow channels with enhanced azimuthally open configurations is a challenge with respect to efficient stacking, deployment, and transport of the gas monitors—a challenge that was overcome by embodiments of the present disclosure.

With reference to FIG. 21, an area monitoring unit 100 may include connections 400, which may be provided in the housing 200. The connections 400 may include an intrinsically safe connection, which may, for example, be used to connect to a solar panel or to a battery pack 1100 to receive power therefrom (e.g., to power supply 300 to power the area monitoring unit 100) in a hazardous environment. An intrinsically safe connection, such as may be provided by at least one connection of connections 400, may eliminate or reduce the risk of triggering, such as by an electrical connection, combustion or ignition of any gasses or fuels in the environment of the area monitoring unit 100. For example, an intrinsically safe connection may prevent the possibility of a spark when an electrical connection is made.

In examples, an area monitoring unit 100 may be stacked (e.g., including with the locking mechanisms as described herein) and safely electrically connected to a battery pack 1100 via the intrinsically safe connection in a hazardous environment that includes a combustible gas. And in examples, an area monitoring unit 100 may be connected to a solar panel while in a hazardous environment to be powered therefrom. This may be a valuable ability, since an area monitoring unit 100 may be intended to operate in environments that could be hazardous (indeed, it may be a function of the area monitoring unit 100 to detect such hazards).

In example embodiments, the connections 400 may include connections, such as power connections, that are not intrinsically safe (e.g., according to a specific industry standard and/or engineering analysis), but that therefore allow for higher power transfer. For example, in a hazardous environment, a user may connect a solar panel or battery pack 1100 directly to the area monitoring unit 100 via the intrinsically safe connection, but such a connection may require a lower amount of power/a lower voltage to be transferred. Thus, if an environment is known to be safe, e.g., there are no hazardous gases present, a solar panel providing a higher voltage/power output (e.g., 12V/60 W) may be directly connected to the area monitoring unit 100 via a connection of connections 400 to power the unit 100 including batteries 310 thereby. In contrast, using the intrinsically safe connection, the solar panel may be limited according to the selected intrinsically safe design, for example limiting voltage and/or current utilization of the area monitoring unit.

In example embodiments, connections 400 may include a universal serial bus (USB) connection, which may, for example, be used to upgrade firmware of the area monitoring unit 100 (e.g., as may reside on the core module 110 or elsewhere), and/or as may be used as a data interface for the area monitoring unit 100 for functions as may be described herein and/or as may be apparent to one skilled in the art upon review of this disclosure. The USB connection, possibly together with all or some other connections of the connections 400, may be together or individually located under one or more removeable face plates and/or doors on the housing 200 of the area monitoring unit 100.

An area monitoring unit 100 according to example embodiments may connect to other area monitoring units 100, to a gateway, and/or to a remote system such as a cloud server. Such connections may take place wirelessly (e.g., with WiFi or Bluetooth), via a cellular connection, via any of the interfaces 400 as described herein, and/or via connections that would be apparent to one skilled in the art upon review of this disclosure.

In an example, the area monitoring unit 100 may serve as a relay for data between the remote system (e.g., via a cellular connection) and sensing devices (e.g., other area monitoring units 100 but not limited thereto) that do not have a cellular connection. Thus, the area monitoring unit 100 may upload its own readings to the remote system as well as those of other devices, while those other devices (including area monitoring units 100) may be placed in the ideal locations for hazardous sensing, rather than a suboptimum location mandated by the need for a cellular signal and/or a WiFi signal to a fixed location device such as a router, hub, server, or the like. Additionally, by knowing both protocols, the area monitoring unit 100 may be able to communicate with both prior and current generations of monitoring units, thereby “bridging the gap” between them to provide successful communication between the generations as well as the remote system, promoting backward compatibility, and/or allowing for different monitors n different network types to interact.

An area monitoring unit 100 according to example embodiments may have visual and/or audio alarms of its own. However, an area monitoring unit 100 may additionally or alternatively trigger external alert equipment, such as a siren, horn, or other infrastructure of the site, to turn on additional alarms. Additionally, an area monitoring unit 100 may act as a relay by allowing other monitoring units (e.g., connected peer devices) to cause it to trigger such alert equipment.

In example embodiments, connections 400 may include a subscriber identify module (SIM) slot that is accessible for a user to provide a user-supplied SIM card for cellular connection of the area monitoring unit 100 therethrough, for example, via a cellular module as described herein.

In example embodiments, an area monitoring unit 100 may connect to a remote system such as a cloud server via a cellular connection or via its connection to other area monitoring units, one or more of which may serve as a gateway or “leader node” within a peer group of area monitoring units. The leader node may serve as the communication link to the remote server, for example via a cellular connection. For example, a certain area monitoring unit 100 may not have cellular access due to its location, but may communicate wirelessly with another area monitoring unit (such as a leader node), which does have cellular access, where such other area monitoring unit may serve as the communication relay between the certain area monitoring unit 100 and the remote system. Such a configuration may help to ensure that communications are sent and received between each area monitoring unit and the remote system. Indeed, any area monitoring unit 100 according to example embodiments may function as such a gateway provided it has appropriate cellular or other coverage.

An example system includes a plurality of gas monitors, each gas monitor including a means for variable ambient air flow accommodation and a means for stacking groups of the plurality of gas monitors. Without limitation to any other aspect of the present disclosure, example means for variable ambient air flow accommodation include any aspect of the present disclosure setting forth flow channels and/or a flow management portion of a gas monitor. Without limitation to any other aspect of the present disclosure, example means for stacking groups of the plurality of gas monitors include any aspects of the present disclosure setting forth stacked monitor interactions and support, weight distribution management of a gas monitor, and/or shaping of a gas monitor for considerations of stacking, storing, and/or transporting groups of gas monitors. The example system further includes a means for operating the plurality of gas monitors as a cooperative monitoring group, and for providing at least selected gas monitoring data to a remote device (e.g., a cloud server). For example, certain operations of the cooperative monitoring group may include watching alerts, getting monitoring data, turning monitors on/off, obtaining status information, obtaining faults, and the like.

In the example system, each gas monitor may further include a multi-threaded power interface. The gas monitor may be configured to operate from any one of the power threads of the multi-threaded power interface. The multi-threaded power interface may include three power threads, i.e., power could be provided through any of the three slots. In certain embodiments, each power thread may include a battery pack slot. The battery pack slot may be accessible while the gas monitor is in a deployed position. In certain embodiments, each power thread may include an external power pack. The external power pack could have the same footprint as, and/or be stackable with, the gas monitors. In some embodiments, the deployable configuration of the gas monitor is a paired gas monitor/external power pack configuration that is deployed stacked. In certain embodiments, each power thread may include a solar connection interface.

In certain embodiments, the means for operating the plurality of gas monitors may further include a means for powering the cooperative monitoring group without intervention for at least 25 days.

In certain embodiments, the means for operating the plurality of gas monitors may further include at least one of: a mesh network communication means between the plurality of gas monitors, a cellular network communication means, or a satellite network communication means.

In certain embodiments, the means for stacking groups of the plurality of gas monitors includes a means for stacking in stacks of between two and eight gas monitors, inclusive.

An example system includes a plurality of gas monitors, each gas monitor including a housing defining a first flow channel and a second flow channel, wherein the first flow channel comprises a first flow path axis and wherein the second flow channel comprises a second flow path axis, and wherein the first flow path axis intersects the second flow path axis at an angle of at least 60 degrees. The example system includes a gas sensing element fluidly coupled to both of the first flow channel and the second flow channel. The example system includes a gas monitor controller configured to control sensing, alerting, and communication operations of the respective gas monitor. The example system includes a remote computing device at least selectively communicatively coupled to at least one of the plurality of gas monitors. The gas monitor controllers and the remote computing device are configured to operate the plurality of gas monitors as a cooperative monitoring group. The housing further defines a sensor assembly, a cellular capable transceiver, and a battery assembly including a plurality of slots configured to each receive a battery pack, wherein the respective gas monitor is configured to operate normally with power provided by a single battery pack. The housing further includes a support assembly at a first end, the support assembly including a plurality of extended members, and an engagement assembly at a second end, the engagement assembly including a plurality of receiving slots matched to engage the plurality of extended members, wherein a geometry of the support assembly and the engagement assembly are configured such that a center of mass of two stacked gas monitors is positioned within a footprint of the plurality of extended members of the support assembly of a lower one of the two stacked gas monitors. In certain embodiments, the gas monitor controllers and the remote computing device are further configured to operate the plurality of gas monitors as the cooperative monitoring group by performing at least one of: sharing external communication resources for communications with the remote computing device, operating the gas monitors as a mesh network, and bridging an external network through at least one of the gas monitors, wherein the bridging connects a main external network to at least one target end point of the external network. Sharing external communication resources for communications with the remote computing device can include, for example: scheduled power management of devices (e.g., leveling power consumption), reduction of total communication cost, assisting devices that are low in power or external connectivity, ensuring that alerts are provided to the remote device, rationality checks, redundancy for important communications, and the like. Operating the gas monitors as a mesh network enables creation of an ad hoc network out of deployed devices without any infrastructure or support other than that the monitors need to each be close enough to adjacent monitors to keep the group connected. In certain embodiments, a connection could be bridged through the remote device (e.g., two deployed groups not quite close enough can still cooperate through the remote device). Bridging an external network (e.g., a group of handheld gas monitors) through at least one of the gas monitors can include active bridging (e.g., the area monitor acts like a member of the external network) or passive bridging (e.g., the area monitor passes messages through without looking at them, or potentially even being able to look at them). In this embodiment, bridging could be through the remote device (e.g., when the bridging area monitor cannot reach the main external network directly).

An example gas monitor includes a housing defining a first flow channel and a second flow channel, wherein the first flow channel comprises a first flow path axis and wherein the second flow channel comprises a second flow path axis, and wherein the first flow path axis intersects the second flow path axis at an angle of at least 60 degrees, a gas sensing element fluidly coupled to both of the first flow channel and the second flow channel, and a gas monitor controller configured to control sensing, alerting, and communication operations of the gas monitor. A remote computing device may be at least selectively communicatively coupled to the gas monitors, wherein the gas monitor controller and the remote computing device are configured to operate the gas monitor as a standalone monitor or as part of a cooperative monitoring group.

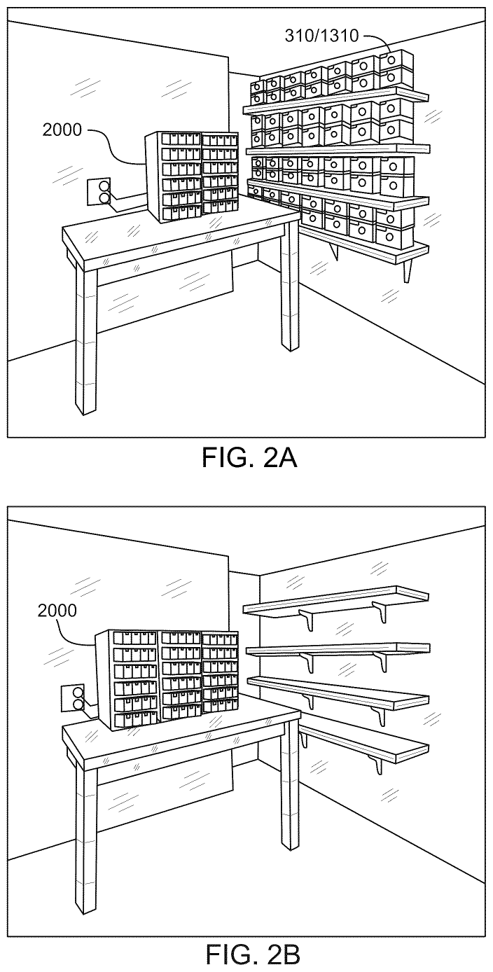

With reference to FIGS. 2A-2B, in example embodiments, a centralized bulk charger 2000 may charge up to 15-20+ batteries 310 or batteries 1310 (e.g., batteries for an area monitoring unit 100 or a battery pack 1100) off of a single electrical outlet. The ability to charge bulk numbers of batteries may permit an increase in the number of batteries usable by an entity, which, in some cases, may allow the entity to rely on fewer area monitoring units 100. A bulk charger 2000 may also charge batteries 310/1310 within a unit/battery pack. Thus, while batteries 310/1310 and/or the units 100/battery packs 1100 that they are contained within are stored, such devices may be charged by the bulk charger 2000.

With reference to FIG. 3, example embodiments may relate to transportation and deployment of area monitoring units 100. For example, there may be a desire to get units 100 out into the field quickly. With the compact, square footprint provided by example area monitoring units 100, which may stack and lock together to secure them, more units 100 may fit into a transportation device such as a vehicle and be thereafter carried or carted (e.g., using a wagon, a dolly, or hand carried) to the deployment location. This may simplify and reduce the deployment time of the units 100. In some examples, a top area monitoring unit 100 of a plurality of stacked units may be provided with a handle for easier carrying. Additionally, as discussed herein, the top and/or all area monitoring units 100 may be provided with an attachment mechanism such as a D-ring for hoisting. In some examples, an area monitoring unit 100 may weigh around 8 pounds, such that a reasonably fit person may carry more than one stacked area monitoring unit 100 for faster deployment of the same.

With reference to FIG. 4, and as described elsewhere herein, area monitoring units 100 may be provided with different mounting solutions to be mounted on beams, columns, tripods, walls, scaffolding, etc. These mounting solutions may allow the units 100 to be as close to work areas as possible without causing falling/tripping hazards, and without being sub-optimally placed owing to awkward and/or larger designs of related art. Indeed, the units 100 may be optimally placed with regard to both horizontal and vertical directions in locations that are not in the way of workers. However, owing to the gas path 230 described by example herein, the area monitoring unit 100 may not need to be placed with regard to a certain orientation or position, as gas in the environment should reach the sensors therein regardless.

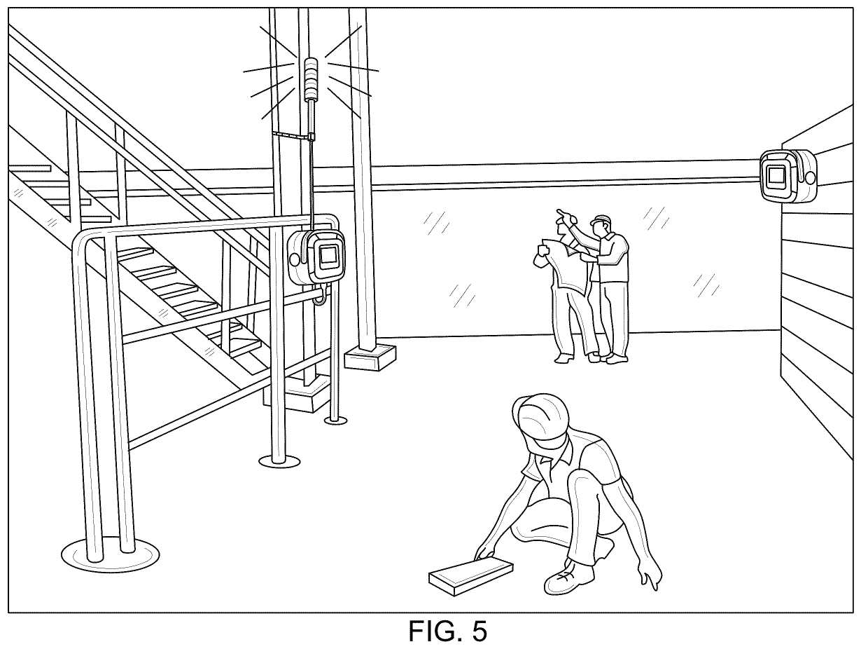

With reference to FIG. 5, and as described elsewhere herein, an area monitoring unit 100 may trigger external hardware—in this case, a flashing light—to alert workers of a dangerous condition in an environment that it is actively monitoring. For example, the sensing module 114 of a monitoring unit 100 may be actively monitoring the work area and detect a dangerous gas while a worker is performing their work order. In addition to other alert steps taken by unit 100, the unit 100 may relay a switch to cause alarms/sirens to go off and provide increased awareness of the dangerous condition. For example, the unit 100 may include one or a plurality of auxiliary relays with configurable alarms and/or thresholds for triggering such alerts on, e.g., external devices. In some embodiments, the area monitoring unit 100 may relay any external device command or communication, such as an external device command or communication that triggers an action or state of any external device (e.g., fan, cooling pump, humidity/spray-down system). In some embodiments, the area monitoring unit 100 may trigger external hardware, such as the flashing light or any other external device, as an indication of a state of a networking capability or a state of network activity. In some embodiments, an aspect of the area monitoring unit 100 itself, such as an onboard light or audio indication, may be triggered as an indication of a state of a networking capability or a state of network activity. In some embodiments, an area monitoring unit 100 may be configured to trigger external devices as described herein based on any device in the unit 100—for example, any sensor of sensing unit 114, any alarm type that the unit 100 provides or that is provided from another area monitoring unit 100 connected thereto, etc.

With reference to FIG. 6, example embodiments may permit easy field maintenance of an area monitoring unit 100. For example, the batteries 310 and/or core module 110 may be replaced in the field without the need to remove the entire area monitoring unit 100. For example, as shown in FIG. 6, there may be no need to remove the unit 100 from the post to which it is attached, or even to power down the unit 100, as one or more of the batteries 310 is replaced.

Certain example aspects of embodiments of the present disclosure are described following. The described aspects are provided to illustrate certain aspects of embodiments of the present disclosure. A given embodiment may include one or more of, or all of, the described aspects.

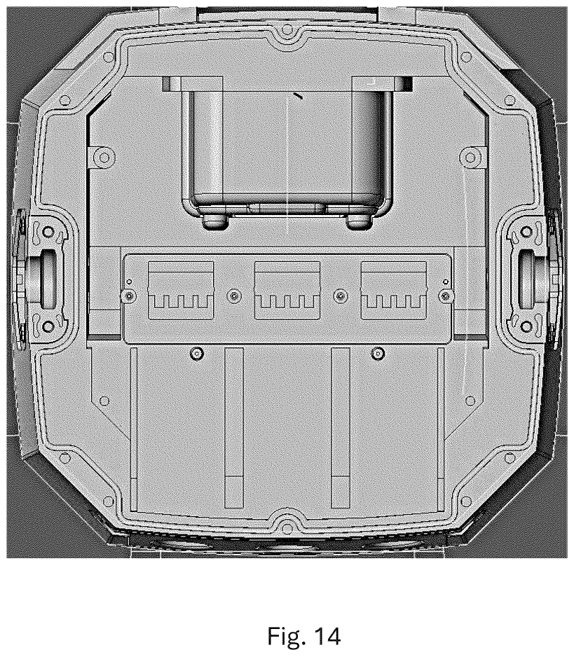

Referencing FIG. 1, an example embodiment depicts two stacked area gas monitors. Referencing FIGS. 2A and 2B, an example embodiment schematically depicts a battery storage and charging assembly. Referencing FIG. 3, an example embodiment depicts selected deployment operations. Referencing FIG. 4, an example embodiment depicts a number of example mounting locations for area gas monitors. Referencing FIG. 5, an example embodiment depicts an alarm operated by an area gas monitor. Referencing FIG. 6, an example embodiment depicts selected battery and/or core replacement operations. Referencing FIGS. 7-13, several views of an example area gas monitor 100 are depicted. Referencing FIG. 14, a bottom view of an example body for an area gas monitor is depicted, consistent with a view of the embodiment of FIGS. 7-13 with the bottom portion 180 removed and/or not present. Referencing FIG. 15, an example bottom portion 180 and/or tray is depicted.

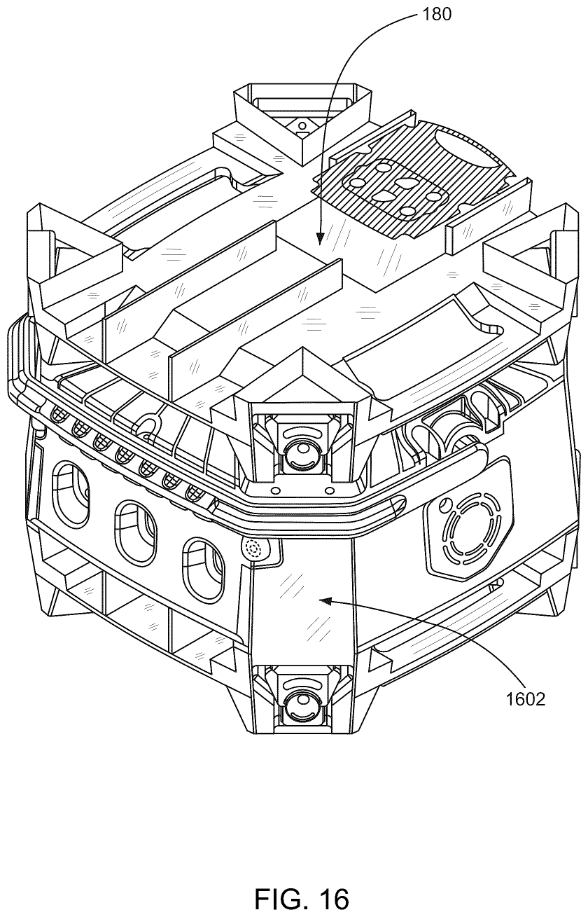

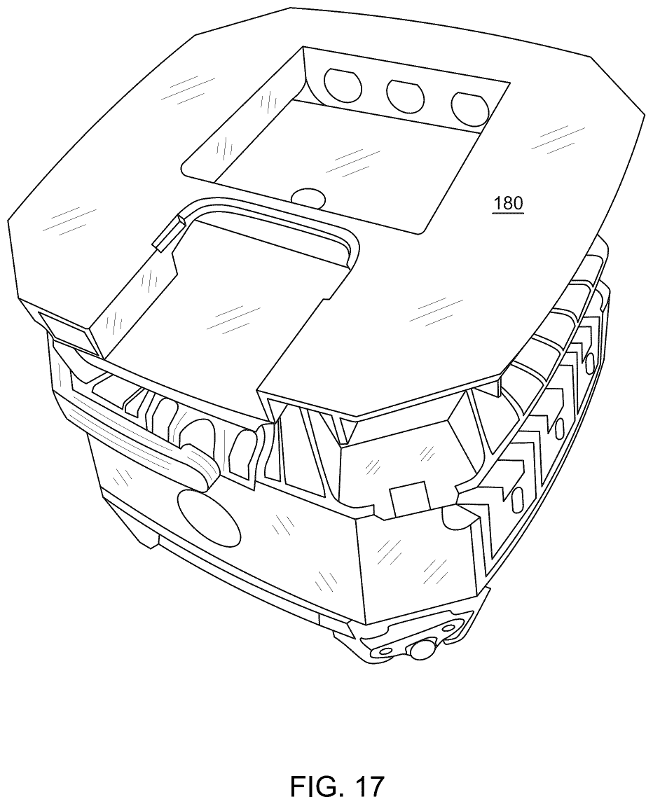

Referencing FIG. 16, an example area gas monitor 1602 is depicted engaged with a bottom portion 180 of another monitor (not shown). Referencing FIGS. 17-19, further views consistent with the embodiment of FIG. 16 are depicted. FIG. 18 depicts the area gas monitor 1602 with the battery faceplate 250 removed and three batteries 310 exposed, for example for service, maintenance, or the like. FIG. 19 depicts an example latching mechanism, and the side of the area gas monitor 1602 having the core module 110 inserted therein. Referencing FIG. 20, an example embodiment consistent with FIG. 16, having the bottom portion 180 of the other monitor separated from the area gas monitor 1602. In the example of FIG. 19, the instrument faceplate 226 and the battery faceplate 250 (removed, in the example) are visible.

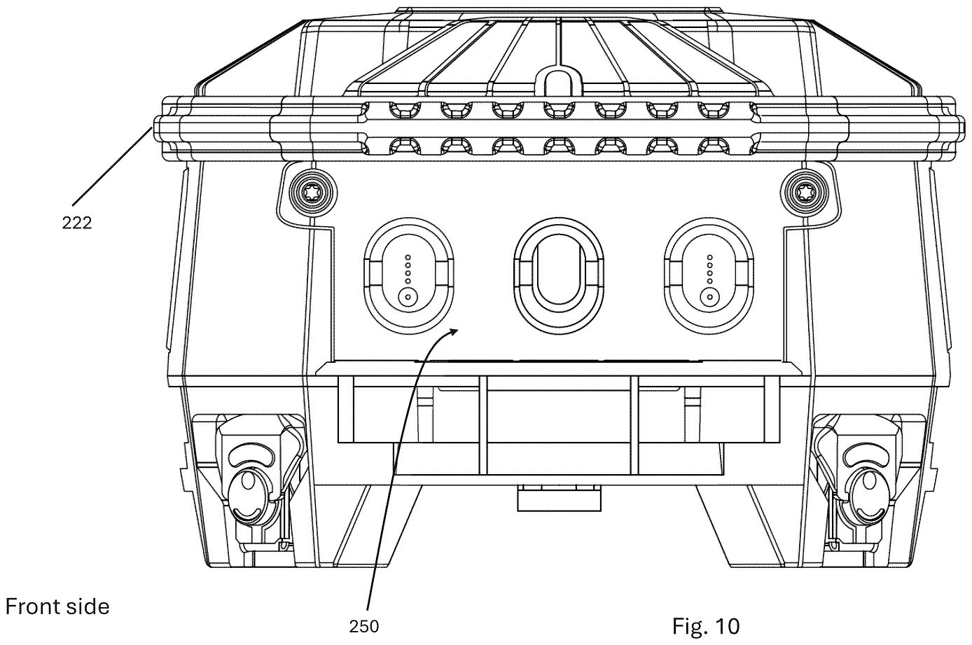

Referencing FIGS. 7-13, a number of views of an example area gas monitor are depicted. FIG. 7 depicts a perspective view with the battery faceplate 250 visible on the left side and the instrument display visible on the top side. FIG. 10 depicts a side view looking at the battery faceplate 250/power connection side of the example monitor. FIG. 11 depicts a side view looking at the core module access side of the example monitor. FIGS. 8-9 are side views depicting the other two sides of the example monitor (e.g., the sides that do not have either the battery faceplate 250 or the core module access). FIG. 12 is a top view of the example monitor with the instrument display visible, and the mounting locations for a covering faceplate 226. FIG. 13 is a bottom view of the example monitor.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

The use of the terms “a” and “an” and “the” and similar referents in the context of describing the disclosure (especially in the context of the following claims) is to be construed to cover both the singular and the plural, unless otherwise indicated herein or clearly contradicted by context. The terms “comprising,” “having,” “including,” and “containing” are to be construed as open-ended terms (e.g., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. The term “set” may include a set with a single member. No language in the specification should be construed as indicating any non-claimed element as essential to the practice of the disclosure.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiment, method, and examples, but by all embodiments and methods within the scope and spirit of the disclosure.

1.-41. (canceled)

42. A system, comprising:
a plurality of gas monitors, each gas monitor comprising:
a means for variable ambient air flow accommodation; and
a means for stacking groups of the plurality of gas monitors; and
a means for operating the plurality of gas monitors as a cooperative monitoring group, and for providing at least selected gas monitoring data to a remote device.

43. The system of claim 42, wherein each gas monitor further comprises a multi-threaded power interface, wherein the gas monitor is configured to operate from any one of the power threads of the multi-threaded power interface.

44. The system of claim 43, wherein the means for operating the plurality of gas monitors further comprises a means for powering the cooperative monitoring group without intervention for at least 25 days.

45. The system of claim 43, wherein each power thread comprises a battery pack slot.

46. The system of claim 45, wherein the battery pack slot is accessible while the gas monitor is in a deployed position.

47. The system of claim 42, wherein the remote device comprises a cloud server.

48. The system of claim 42, wherein the means for operating the plurality of gas monitors further comprises at least one of:
a mesh network communication means between the plurality of gas monitors;
a cellular network communication means; or
a satellite network communication means.

49. The system of claim 42, wherein the means for stacking groups of the plurality of gas monitors comprises a means for stacking in stacks of between 2 and 8 gas monitors, inclusive.

50. A system, comprising:
a plurality of gas monitors, each gas monitor comprising:
a housing defining a first flow channel and a second flow channel, wherein the first flow channel comprises a first flow path axis and wherein the second flow channel comprises a second flow path axis, and wherein the first flow path axis intersects the second flow path axis at an angle of at least 60 degrees;
a gas sensing element fluidly coupled to both of the first flow channel and the second flow channel; and
a gas monitor controller configured to control sensing, alerting, and communication operations of the respective gas monitor;
a remote computing device at least selectively communicatively coupled to at least one of the plurality of gas monitors; and
wherein the gas monitor controllers and the remote computing device are configured to operate the plurality of gas monitors as a cooperative monitoring group.

51. The system of claim 50, wherein each of the plurality of gas monitors further comprises:
wherein the housing further defines a sensor assembly;
a cellular capable transceiver; and
a battery assembly comprising a plurality of slots configured to each receive a battery pack, and wherein the respective gas monitor is configured to operate normally with power provided by a single battery pack.

52. The system of claim 51, wherein each of the plurality of gas monitors further comprises:
wherein the housing further comprises:
a support assembly at a first end, the support assembly comprising a plurality of extended members; and
an engagement assembly at a second end, the engagement assembly comprising a plurality of receiving slots matched to engage the plurality of extended members; and
wherein a geometry of the support assembly and the engagement assembly are configured such that a center of mass of two stacked gas monitors is positioned within a footprint of the plurality of extended members of the support assembly of a lower one of the two stacked gas monitors.

53. The system of claim 50, wherein the gas monitor controllers and the remote computing device are further configured to operate the plurality of gas monitors as the cooperative monitoring group by performing at least one operation selected from the operations consisting of:
sharing external communication resources for communications with the remote computing device;
operating the gas monitors as a mesh network; and
bridging an external network through at least one of the gas monitors, wherein the bridging connects a main external network to at least one target end point of the external network.

54.-68. (canceled)
